(19) United States
(12) Patent Application Publication
(10) Pub. No.: US 2003/0148521 A1
(43) Pub. Date: Aug. 7, 2003

(54) Conditionally replicative and conditionally active viruses

(76) Inventors: John C. Bell, Ottawa (CA); David F. Stojdl, Ottawa (CA); Douglas A. Gray, Ottawa (CA); Nahum Sonenberg, Montreal (CA); Brian Lichty, Russell (CA)

Correspondence Address: Mr. Adrian Zahl, Ridout & Maybee LLP, 19th Floor, 150 Metcalfe Street, Ottawa, ON, K2P 1P1, CA

(21) Appl. No.: 10/115,987

(22) Filed: Apr. 5, 2002

Related U.S. Application Data
(60) Provisional application No. 60/281,781, filed on Apr. 6, 2001.

(51) Int. Cl. C12N 15/86; C12N 7/00

(52) U.S. Cl. 435/456; 435/235.1

(57) ABSTRACT

Viral gene constructs and modified viruses contain properties which permit them to replicate or have activity only in target cells such as diseased or otherwise infected cells.

### FIELD OF THE INVENTION

The invention relates to viral gene constructs and genetically-modified viruses which are able to replicate and/or have cytotoxic activity only in target cells. The invention relates to kinase-dependent viruses as well as protease restricted virus strains, and other viral constructs which are conditionally replicative or engineered to conditionally deliver a therapeutic gene product.

### BACKGROUND OF THE INVENTION

Viruses contain only limited genetic information and thus rely upon host cell machinery for the synthesis, regulation and modification of many of their gene products. By tailoring viral genomes, it is possible to direct the replication of viral genomes in particular cell types. As an example, certain recombinant strains of adenovirus (Cancer Res. Jan. 15, 2000; 60(2): 334-41) contain host cell promoter elements that determine in which cell or tissue types viral genes can be expressed.

The present invention relies on certain properties of viruses to target diseased cells. The types of viruses which form the subject of this invention and the strategies for employing such viruses, include:

Kinase and phosphatase regulated viruses

Protease restricted viruses

Viruses regulated by the ubiquitin degradative pathway

Viral genomes that incorporate eukaryotic or viral translational regulatory sequences

Viruses that incorporate a combination of translational and post-translational regulatory sequences

Viruses which conditionally replicate or express gene products in target cells

Protein Kinases and Virus Replication

In certain diseased cells (e.g. cancer cells) specific protein kinases are commonly de-regulated. Many viral gene products undergo post-translational phosphorylation and yet few if any viral genes encode enzymes capable of this activity. Rather, viral phosphorproteins appear to have evolved to become good substrates of host kinases and phosphatases. In general the host enzymes (kinases and phosphatases), used by viruses are ubiquitously expressed thus allowing the virus to grow in many tissue types. It is an object of this invention, to tailor viral phosphoproteins so that they are efficiently modified/regulated by kinases/phosphatases that are specific to certain diseased cells.

Viral Proteases

A strategy widely used by several distinct types of viruses to regulate gene expression is modification of proteins by viral specific proteases. Examples include but are not restricted to viral proteases encoded by poliovirus, herpes virus, hepatitis C virus, Dengue virus, Coxsackie virus, adenovirus and retroviruses (including human immunodeficiency virus (HIV) and T cell Leukemia virus (HTLV)). Given that proteolysis is such a critical step in viral reproduction it seems likely that viral proteases should be good targets for the identification of antiviral compounds. Indeed novel HIV protease inhibitors are being used clinically (J. Virol. May 2000; 74(9), 4127-38). Instead of inhibiting viral proteases, an alternative therapeutic strategy prepared by the present inventors is to use the specific expression and activity of viral proteases to activate therapeutic viruses.

Ubiquitin Degradative Pathway

Ubiquitin (Ub) is a small protein which is covalently ligated to proteins targeted for degradation. A series of proteins or protein complexes mediate these regulated events, including the E1 ubiquitin activators, E2 ubiquitin conjugation enzymes and the E3 ubiquitin ligases (reviewed in Current Biology 1999 9;R554-R557). Many components of the Ub pathway function and/or are expressed in a tissue specific (Genes Cells November 1998; 3(11) 751-63), disease specific (J. Cell. Biochem. Suppl. 2000; 34:40-51; Proc. Natl. Acad. U.S.A. Jan. 30, 2001; 98(3) 1218-1223; Proc Natl. Acad. Sci. U.S.A. Oct. 26, 1999; 96(22): 12436-41) or stress specific manner (Oncogene Sep. 17, 1998; 17(11 Reviews): 1483-90; J. Biol. Chem. Jun. 9, 2000; 275(23): 17229-32; Nat Cell. Biol. July 2000; 2(7) E121-3).

It is proposed by the present inventors to modify a viral genome to permit the virus to target specific cells or otherwise carry out a function, subsequent to the virus being acted on by a component of the Ub pathway.

Transiational Regulatory Sequences

A further aspect of this invention relates to the discovery of mRNA sequence elements that control the translation of such mRNA. This invention provides a means to exploit these regulatory elements to govern the control of replication of a virus. Many viruses, especially those that encode their own nucleic acid polymerases and replicate in the cytoplasm of infected cells overcome host cell restrictions by usurping the host translational machinery. These viruses have adopted several different strategies for controlling protein translation including stealing of mRNA cap structures; inhibition of cap dependent translation and inactivation of the cellular PKR enzyme. Indeed there are several examples of mutant viruses that are impaired in their ability to efficiently replicate due to restrictions on the translation of viral mRNAs. Furthermore, there are examples of modest increases in translation efficiency (2 fold) causing a change in cellular tropism of certain viruses. The present invention has as an object to provide a genetically modified virus which incorporates sequence elements into its mRNA which controls the translation of viral mRNAs in a target cell specific fashion.

Types of Translation Regulatory Elements Found in Cellular mRNAs

Structured 5UTRs. While the majority of cellular mRNAs contain relatively short leader sequences (5′UTR of 100 nucleotides), it is clear that a select group of mRNAs contain complex structured long 5′UTRs. Often these mRNAs are poorly expressed in resting cells but in growing or malignant cells have robust translation (Int. J. Biochem. Cell Biol. January 1999; 31(1)73-86). A common feature of malignant cells is the over expression of elF-4E. Over expression of elF-4E leads to increased unwinding of complex 5′UTR elements and enhanced translation. Cellular mRNAs with complex 5′UTRs that have increased translation in the presence of excess elF-4E include c-myc, ornithine decarboxylase and ornithine aminotransferase (Int. J. Biochem. Cell Biol. January 1999; 31(1):59-72).

Internal Ribosome Entry Sites: While most cellular mRNAs are translated by a cap dependent mechanism, there are notable exceptions. For example the ornithine decarboxylase (ODC) mRNA, the c-myc mRNA, the XIAP mRNA and many others have IRES elements which facilitate cap independent translation (Cell, Apr. 28, 2000; 101(3):243-5). There are numerous examples of IRES elements being responsible for protein translation in malignant cells (Oncogene. Sep. 7, 2000; 19(38)). Additionally cell cycle translation or translation in a stressed cell environment may be under the control of certain IRES elements.

5′ Terminal Oligopyrimidine Tracts. Translation of mRNAs for ribosomal proteins are regulated in a growth dependent fashion due to the presence of 5′ oligopyrimidine tracts (5′-TOPS). These TOPS are usually 5-14 consecutive pyrimidines found at the beginning of short (40 nucleotides or less) UTRs (Eur. J. Biochem. November 2000; 267(21):6321-30).

Upstream Open Reading Frames (uORFs): Certain mRNAs contain open reading frames upstream of the initiator methionine. These uORFs are frequently found in growth promoting genes and dictate the efficiency of mRNA translation. Some uORFs appear to regulate the usage of downstream re-initiation of translation (J. Biol. Chem. Apr. 17, 1998; 273(16):9552-60).

mRNA Stability Sequences: Particular mRNA sequences are recognized as targeting signals for RNA degrading enzyme complexes. In general these contain AU rich sequences are found in 3′ untranslated regions (Mol. Cell Biol. Feb. 1, 2001; 21(3):721-730).

These codons found in the 5′UTR can act in concert with 3′UTR sequences to dictate the efficiency of usage. In malignant cell lines; the efficiency of CUG codon usage in FGF-2 mRNA is drastically up regulated (J. Biol. Chem. Jun. 23, 2000; 275(25):19361-7).

Transiational Repressor Elements: Certain cis acting elements found in the 3′ and 5′UTR regions of cellular mRNAs can be conditionally activated by binding of protein factors. Examples include sites found in the p53 and thymidytate synthase mRNAs. In both cases, the protein encoded by the cognate mRNA binds the transtational repressor element sequence. As a result the translation of them mRNA is negatively regulated by the protein it encodes (Oncogene. Nov. 11, 1999; 18(47):6419-24).

### SUMMARY OF THE INVENTION

This invention relates to novel genetically modified viruses that productively infect specific targeted cell types. It is an objective of this invention to create viruses that will function as replicating therapeutics. Such recombinant viruses are limited in their ability to grow or have cytotoxic effect in normal cell types but have enhanced or exclusive replication or cytotoxic effect in diseased or damaged cells. Without wishing to be bound to specific properties of such constructs, these conditionally replicating viruses may deliver therapeutic gene products to diseased cells or upon replication lead to the killing and elimination of diseased cells. These viruses may also be engineered to deliver, in a conditional fashion, therapeutic molecules either to protect or kill the target tissue.

The present invention describes recombinant viruses that only function in specific target cells. These target cells provide the unique translational or post-translational conditions required by said recombinant virus in order to replicate. These recombinant viruses are disabled in non-target cells as these cells lack the unique translational or post-translational conditions upon which said recombinant viruses are dependent.

The term “viral replication” refers to any aspect of the viral life cycle and is not limited to genomic replication.

As further described herein, a particular aim of this invention is to describe recombinant viruses which differ from their wildtype progenitors in that they have been modified such that they depend on different and unique translational and/or post-translational regulation of their viral proteins. These modifications include, but are not limited to those that 1) place special restrictions on the translation of viral messenger RNA such that these messages are only translated in a particular host cell and/or 2) place viral phosphorproteins under the regulation of host cell kinases in such a manner so as to determine which host cells these viral phosphoproteins are functional in and/or 3) place novel viral polyproteins or proproteins under the regulation of specific proteases present or active only in specific host cells or microenvironments and/or 4) alter the stability of viral proteins such that they are stabilized/destabilized in particular host cells by virtue of their design.

Desirable target cells which have unique translational and/or post-translational regulatory conditions include, but are in no way limited to malignant cells, virally-infected cells, bacterially-infected cells, cells harbouring intracellular parasites and stressed cells (i.e. hypoxic cells).

According to the invention, a “recombinant/genetically modified virus” is any virus (RNA or DNA) that has been modified from the wildtype such that the translational and/or post-translational regulation of said virus is altered in such a manner so as to restrict the translation and/or function of viral proteins to a particular target cell or cells.

In one aspect, the invention comprises a virus modified to contain in its genome, a viral gene comprising two separate open reading frames (ORFs), a first of said ORFs comprising a fusion with a sequence coding for a recognition sequence for a cellular kinase protein specific to a diseased cell, and a second of said ORFs comprising a fusion with a sequence coding for a protein which binds to said recognition sequence exclusively when said recognition sequence is phosphorylated, thereby reconstituting a viral protein, said reconstituted viral protein being essential to viral replication.

Preferably the viral protein is essential to expression of virally encoded genes or transgenes.

In a further aspect, the invention relates to a genetically modified virus comprising two separate proteins, a first of said a proteins including a fusion with a recognition sequence for a cellular kinase and a second of said proteins including a domain which recognizes said cellcular kinase only when said domain is phosphorylated, said first and second proteins being capable of connection to each other only when phosphorylated by a kinase within a target cell, thereby forming a complex capable of inactivating or killing said target cell.

The invention further relates to a genetically modified virus comprising a gene sequence which codes for a mutated viral phosphorprotein having a phosphorylation site of a type which when non mutated is phosphorylated by a cognate kinase, said phosphorprotein being capable of acting within a target cell such that said viral phosphoprotein is not phosphorylated by said cognate kinase, but rather is recognized solely by a kinase which is either restricted in its expression, or hyperactivated in a target cell, wherein phosphorylation of said viral phosphorprotein by said kinase is critical to viral replication thus restricting the replication of said virus to said target cell.

The invention further comprises a virus modified to contain in its genome, a gene coding for a viral protein fused to an inhibitory domain, said inhibitory domain for preventing the function of said viral protein produced by said virus, unless said domain is phosphorylated by a kinase or a type which is either restricted in its expression or hyperactivated in a target cell.

The invention further relates to a virus modified to contain in its genome an inhibitory protein, said inhibitory protein for inhibiting the function of a viral protein when binding to said viral protein, unless said inhibitory protein is phosphorylated by a kinase which is either restricted in its expression or hyperactivated in a target cell.

The invention further relates to a genetically modified virus containing a plurality of genes coding for a plurality of separate proteins wherein at least two of said genes are fused, forming a fused portion, so as to produce but a single mRNA, said fused portion coding for a polyprotein including a specific protease recognition sequence which when cleaved within a specific protease expressing target cell, generates functional proteins which when separated, and only when separated, allow for replication of said virus. The polyprotein maybe capable upon cleavage of said protease recognition sequence by said specific protease expressed in said target cell, of generating functional proteins which separated, and only when separate, allow for expression of a virally encoded transgene.

The invention further relates to a genetically engineered virus containing a viral gene expressed as a fusion protein including: an inhibitory domain appended to a viral protein, said inhibitory domain for preventing the function of said fusion protein, a specific protease cleavage site between said inhibitory domain and said viral protein sequence for cleavage of said fusion protein by a specific protease contained within a target cell to yield a functional viral protein free to said inhibitory domain. Preferably, the inhibitory domain is for destabilizing said viral protein sequence. The viral protein sequence may include a ubiquitin (Ub) monomer.

The inhibitory domain may be for directing said viral fusion protein to an inappropriate subcellular compartment. Preferably, the inhibitory domain is fused to a viral protein and is for inhibiting said viral protein, said viral protein being critical to viral replication. Preferably, the inhibitory domain is fused to a viral protein and is for inhibiting said viral protein, said viral protein being critical to the expression of a viral transgene.

The invention also relates to a genetically modified virus of the type that when unmodified, has a gene and cleavage sites for a protease, wherein said gene and said cleavage sites are respectively deleted and altered such that said virus is susceptible to a specific heterologous protease produced exclusively in a target cell, said cleavage of said cleavage sites by said heterologous protease being required for the replication of said modified virus.

The invention further relates to a genetically modified virus for infecting a target cell, said target cell overexpressing or uniquely expressing a specific protease, said virus comprising a viral genome having an envelope or surface protein which when proteolytically cleaved by said specific protease, becomes activated for infecting, damaging or killing said target cell. Preferably, the envelope or surface protein is capable of being activated by a metalloproteinase, or the envelope or surface protein is capable of being activated by prostate-specific antigen (PSA).

The invention also covers a virus modified to contain in its genome a transgene, the expression of said transgene being dependent on a host-expressed protease produced by a target cell. Preferably, the transgene is modified to become a substrate for said protease for activation in said target cell expressing said specific protease.

The invention also relates to a genetically modified virus containing a plurality of cistrons, at least two of said cistrons being linked by a nucleotide sequence acting as an internal ribsome entry site (IRES) element, said IRES element being exclusively or preferentially active in a target cell population in such a way that the second of said two linked cistrons is converted to a protein product only in said target cell.

Preferably, the second of said two linked cistrons encodes a protein product critical to the replicative cycle of said virus.

Preferably, the replication of said virus is toxic to said target cell.

Preferably, said second of said two linked cistrons encodes a protein product, said protein product being toxic to said target cell.

Preferably, said second of said two linked cistrons encodes a protein product, said protein product being therapeutic to said target cell.

Preferably, said IRES element is exclusively or preferentially active in dividing cells.

Preferably, said IRES element is derived from the ornithine decarboxylase mRNA 5′ untranslated region.

Preferably, said IRES element is active only in stressed cells, said stressed cells including hypoxic cells.

Preferably, said IRES element is active only in said target cell, said target cell being selected from the group comprising an activated T cell, a tumour cell, or a stern cell.

Said IRES element is active only in said target cell, said target cell being a tumour cell.

Alternatively, said IRES element is active only in cell types excluding normal brain cells.

Said IRES element may be derived from a eukaryotic gene, or a viral gene.

The invention may comprise a genetically modified virus containing a plurality of genes essential to virus replication, at least one of said genes having a CUG initiator codon, said CUG codon for utilization preferentially in a target cell, or alternatively a genetically modified virus containing a plurality of genes, at least one of said genes having a CUG initiator codon, said CUG initiator codon for utilization preferentially in a tumour cell.

The invention may comprise a genetically modified virus containing a plurality of genes essential to virus replication, at least one of said genes having a 5′-terminal oligopyrimidine tract (TOP), said virus being capable of preferential replication in cells that can efficiently translate 5′-TOP containing mRNA. The virus may further contain a gene encoding a toxin, said toxin gene having a 5″TOP.

The invention may comprise a genetically modified virus containing a plurality of genes essential to virus replication, at least one of said genes encoding an mRNA having a sequence comprising a translational repressor element having a sequence, said translational repressor element being preferentially inactive in a target cell. Preferably, said sequence of said translational repressor element is derived from the p53 gene, or said translational repressor is a 66-nucleotide element derived from the p53 gene encoded mRNA. Preferably, said virus is capable of growing exclusively in cells lacking the active wild type p53 gene.

The invention may comprise a genetically modified virus containing a plurality of genes essential to virus replication, at least one of said genes encoding an mRNA having a structured 5′ untranslated region (UTR), said 5′UTR being derived from a mammalian gene and for inhibiting translation in non-target cells, thereby selectively facilitating translation of said mRNA in a target cell.

Said target cell overexpresses eukaryotic initiation factor 4E (elF-4E), or said target cell is a tumour cell.

Optionally, the virus may further contain a gene encoding an mRNA having a structured 5′UTR, said gene encoding a toxin, or further contain a gene encoding an mRNA having a structured 5′UTR, said gene encoding a therapeutic protein.

Said structured 5′UTR may be derived from the human c-myc gene or the human Fgf-2 gene.

The invention may comprise a genetically modified virus containing a plurality of genes essential to virus replication, at least one of said genes encoding an mRNA having an upstream open reading frame (uORF), said uORF for regulating the frequency of usage of downstream initiator codons in said at least one gene. Preferably, said uORF is derived from, or is analogous to the uORF form the C/EBP mRNA.

The invention may comprise a genetically modified virus containing a plurality of genes, at least one of said genes encoding a gene product having an amino acid sequence targeting said virally encoded gene product for degradation by a member of the Ub pathway present in a normal healthy cell, but absent in a target cell having a defective or disrupted Ub pathway.

The invention may comprise a genetically modified cytolytic virus comprising a virus genome encoding an inhibitory of the replication of said virus genome, said inhibitor being subject to degradation by the Ub pathway in a target cell and being stable in a healthy normal cell.

The invention may comprise a genetically modified virus comprising a virus genome coding for a first active toxic protein and a second inhibitor protein, said second inhibitor protein for inhibiting said first toxic protein and said inhibitor protein being selectively degradable by the Ub pathway in a target cell, whilst not being degradable in a healthy cell.

The invention may comprise a genetically modified virus comprising a virus genome coding for an active toxic protein, said toxic protein being selectively degradable by the Ub pathway in a healthy cell, but not in a target cell having a defective or disrupted Ub pathway.

### BRIEF DESCRIPTION OF THE DRAWINGS

FIG. 1A schematically illustrates phosphorylation of a VSV P/NS protein by a ubiquitous host kinase;

FIG. 1B schematically illustrates a recombinant P/NS protein phosphorylated by a target cell specific, non-ubiquitous kinase;

FIG. 2A is an amino acid sequence of a wild type P/NS protein of VSV;

FIG. 2B is an amino acid sequence of a recombinant P/NS protein;

FIG. 2C is an amino acid sequence of a further recombinant P/NS protein;

FIG. 3A is a schematic drawing of a VSV genome and a recombinant VSV genome according to the present invention;

FIG. 3B is a schematic drawing of a polymerase complex in an active and inactive form, both wild type and recombinant according to the present invention;

FIG. 4A is a schematic drawing of a VSV genome, and a further embodiment of a recombinant VSV genome according to the present invention;

FIG. 4B is a schematic drawing of the recombinant viral genome of FIG. 4A, within a healthy cell;

FIG. 4C shows the recombinant genome of FIG. 4A, within an HIV infected cell;

FIG. 5A is a schematic drawing of a further type of VSV recombinant genome, along with a wild type VSV genome;

FIG. 5B schematically illustrates cleavage of ubiquitin/VSV polymerase construct;

FIG. 6 is a schematic drawing showing a VSV construct according to the present invention, and its activity in a target cell and a healthy cell;

FIG. 7 is a further VSV construct according to the invention, and its activity in a healthy cell and a target cell;

FIG. 8 is a schematic drawing of a further embodiment of a VSV according to the invention, and its activity in a healthy cell and a target cell;

FIG. 9 is a schematic drawing of a viral construct for expressing an anti-apoptotic factor and an inhibitor of this same factor, and its activity in a normoxic cell and a hypoxic cell;

FIG. 10A is a schematic drawing of a normal VSV genome, and a recombinant VSV genome according to the invention;

FIG. 10B is a schematic drawing of the construct of FIG. 10A, within a healthy cell and a target cell.

FIG. 11 is an amino acid sequence listing for HIE-1 alpha protein (sequence ID No. 14).

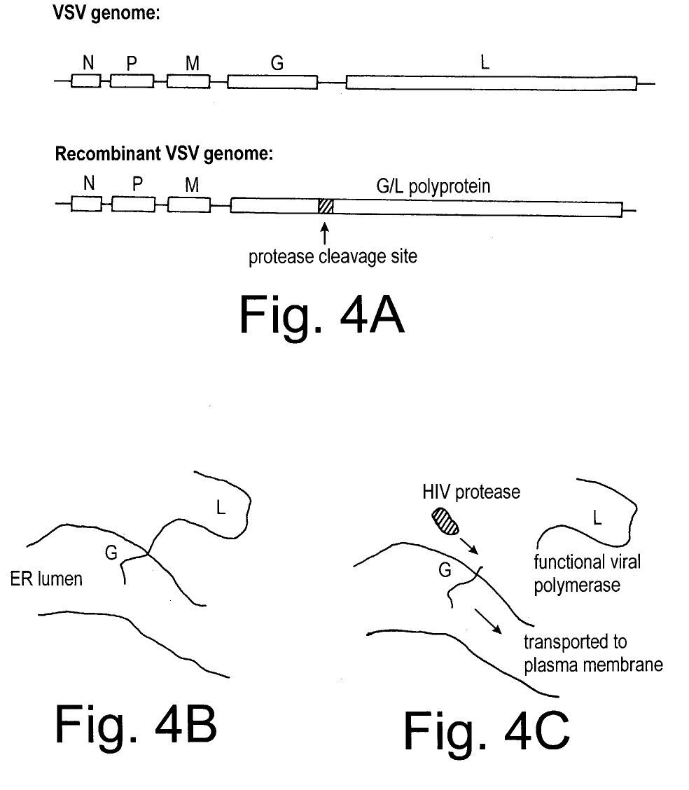

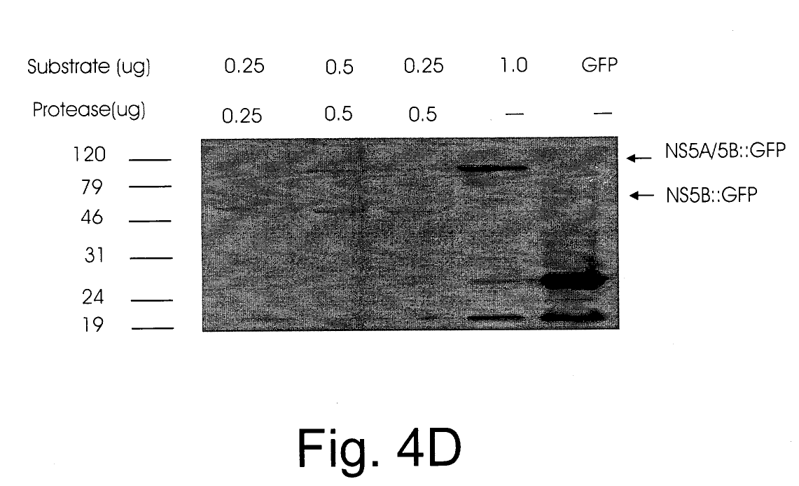

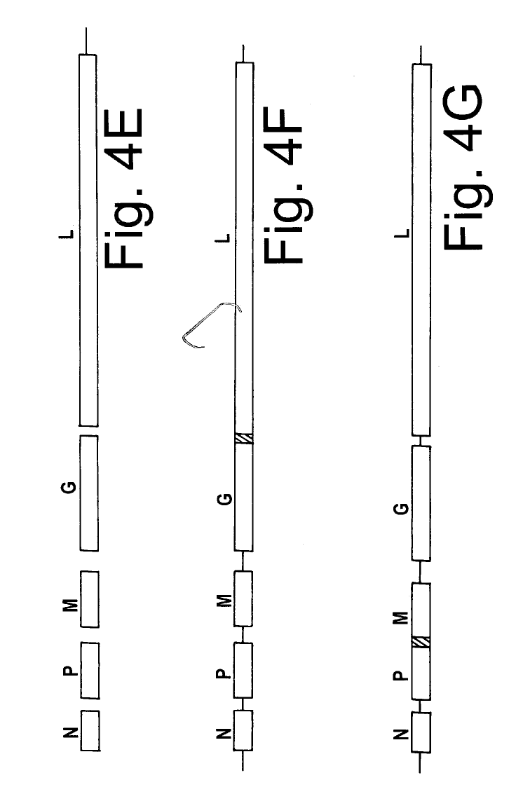

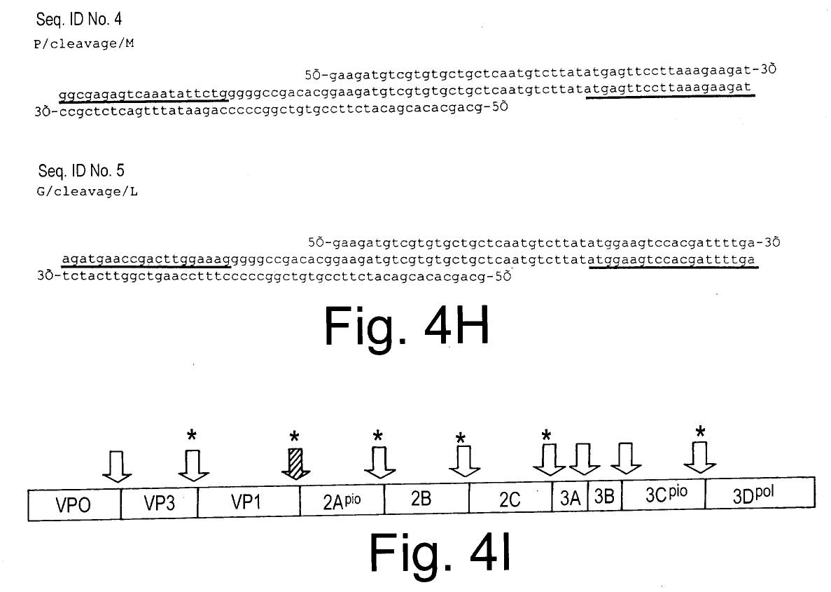

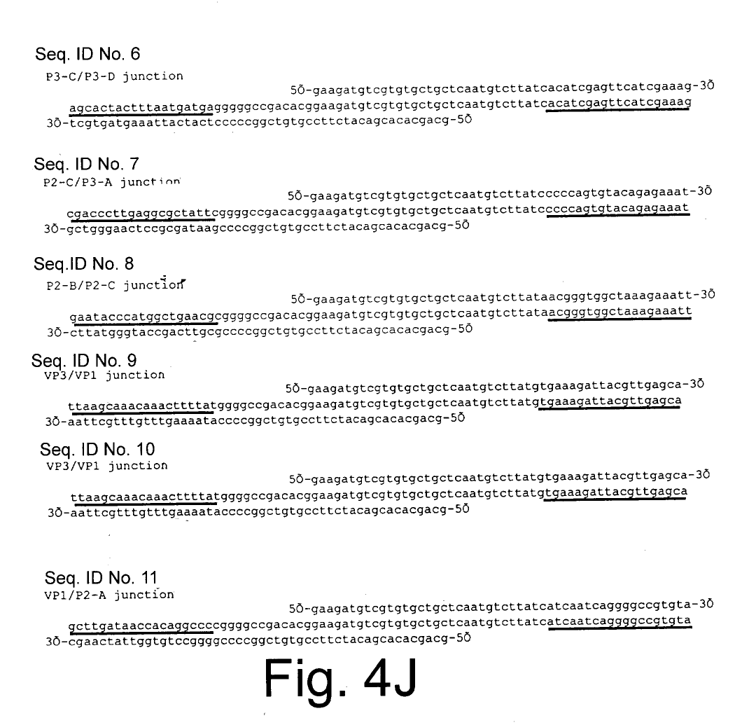

FIG. 4D is a reproduction of a western blot.

FIG. 4E is a schematic drawing of a wild type VSV genome.

FIG. 4F is a schematic drawing of a recombinant VSV genome.

FIG. 4G is a schematic drawing of a further recombinant VSV genome.

FIG. 4H is a DNA sequence for a recombinant VSV genome (sequence ID Nos. 4 and 5).

FIG. 4I is a schematic drawing of coxsackie virus polyprotein.

FIG. 4J comprises six DNA sequences for engineered cleavage sites of the coxsackie protein of FIG. 4I (sequence ID Nos. 6-11).

FIG. 12 is a DNA sequence of an ODDD domain (sequence ID No. 12).

FIG. 13 is a gene sequence of a further ODDD domain (sequence ID No. 13).

### DETAILED DESCRIPTION OF THE PREFERRED EMBODIMENTS OF THE INVENTION

In an embodiment of the invention, the recombinant virus comprises a virus whose replication and/or gene expression is dependent on the phosphorylation of a viral phosphorprotein. This viral phosphoprotein has been modified from the wildtype sequence such that it is specifically recognized by a kinase that is uniquely expressed and/or hyperactive in a particular target cell.

In a further embodiment of the invention, the recombinant virus comprises a virus whose replication and/or gene expression is dependent on the activity of a protease present in a particular target cell or microenvironment. Said recombinant virus encodes a novel polyprotein or proprotein, the cleavage of which by said protease regulates the function of said polyprotein or proprotein.

In a further embodiment of the invention, the recombinant virus comprises a virus whose replication and/or gene expression is dependent on translation of viral messenger RNAs modified such that they are only translated in cells harbouring particular translational conditions. Such modification of viral mRNAs would restrict the replication and/or gene expression of said recombinant virus in desirable target cells.

In a further embodiment of the invention, the recombinant virus comprises a virus whose replication and/or gene expression is dependent on viral proteins that have been modified from the wildtype such that they are stabilized or destabilized in target cells. Said recombinant viral proteins are only able to function in host cells that have particular protein degradation mechanisms active or absent thus restricting the replication and/or gene expression of said recombinant virus.

### EXAMPLES

The present invention and methods are further described in the context of the following examples. These examples serve to illustrate further the present invention and are not intended to limit the scope of the invention.

Viruses Engineered to be Regulated By Protein Kinases

### Example 1

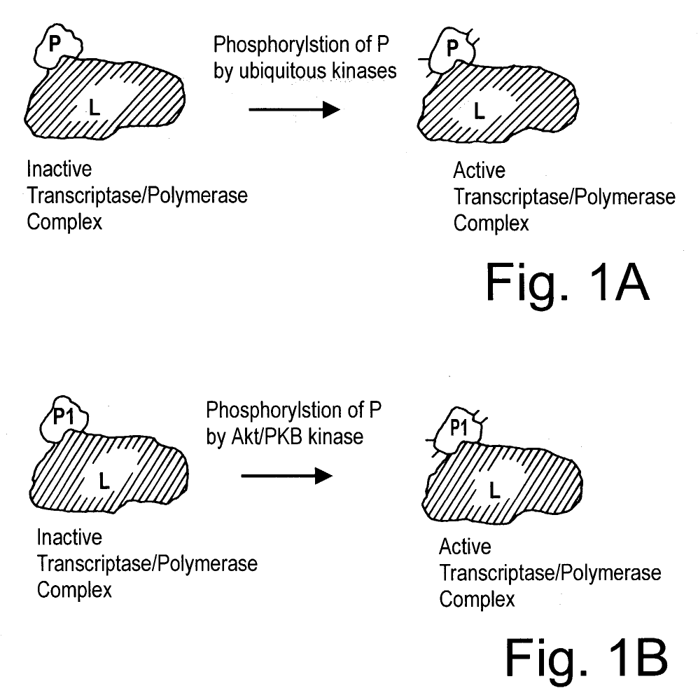

Activated Serine/Threonine Kinases (FIGS. 1A and 1B)

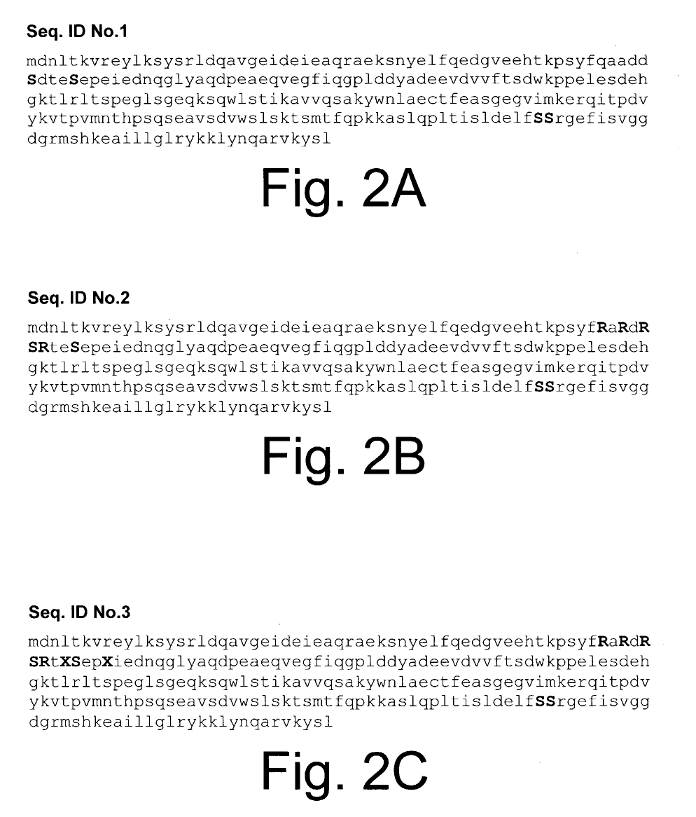

The P protein of vesicular stomatitis virus (VSV) is phosphorylated at several residues that in turn determines the activity of the viral polymerase complex. Phosphorylation of serines at positions 60 and 64 (S60 and S64) is required for efficient viral transcription by the P/L polymerase complex (Pattnaik et al. J. Virol. November 1997; 71(11): 8167-75). Phosphorylation of serines at positions 226 and 227 (S226 and S227) is required for efficient replication of the viral genome by the P/L polymerase complex (Hwang et al. J. Virol. July 1999; 73(7):5613-20). These sites are phosphorylated by ubiquitously expressed host kinases thus permitting replication of the virus in a broad range of cell types. Restriction of the replication of the virus can be attained by mutating the phosphorylation sites in the P protein to an amino acid sequence which is no longer recognized by these ubiquitous kinases but rather is now recognized by a kinase which is restricted in its expression or hyper-activated in certain cell types. Altering kinase specificity for a particular substrate has been demonstrated for the transcription factor c-Jun (EMBO J. Dec. 15, 1994; 13(24):6006-10). In this example, site directed mutagenesis of the c-Jun protein caused it to be regulated by the cAMP dependent kinase PKA instead of its normal cognate kinase JNK. The P protein phosphorylation sites may be altered by site directed mutagenesis to become recognized by a kinases that is over-expressed, mutated or activated in cancer cells (see FIGS. 1 and 2). An example of a kinase of this type would be in the serine/threonine kinase Akt/PKB that is found to be commonly activated in malignant cells (Annu. Rev. Biochem. 1999; 68:965-1014).

### Example 2

Phosphotyrosine/SH2 Domain Interactions

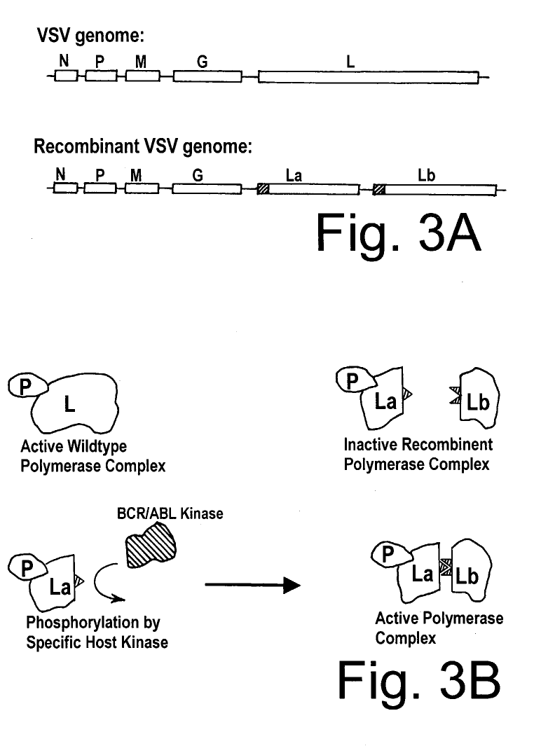

Many proteins contain functionally distinct domains that work in concert to carry out the protein's function. An example would be the VP 16 protein of herpes simplex virus, which contains both a DNA binding domain and an activation domain (Genes Dev. December 1990; 4(12B):2383-96). The gene coding for a viral protein can be split in to 2 separate genes contained in a single recombinant genome. One portion of the viral protein (domain A) would be expressed as a fusion between the viral protein and the recognition sequence/phosphorylation target of a particular cellular kinase. The other portion (domain B) of the viral protein would be expressed as a fusion with a domain which binds to the above recognition sequence but only when it is phosphorylated (i.e. the target for a cellular tyrosine kinase on one portion would be recognized by the appropriate SH2 domain fused to the other portion: see FIG. 3). In the presence of the cellular kinase the phosphorylation target on domain A would become phosphorylated and then bound by domain B thereby reconstituting a functional viral protein. This will restrict the activity of this viral protein to those cells where a particular kinase is functioning. An example of an activated kinase that could be exploited in this fashion is the BCR-ABL kinase, an enzyme that only exists in certain types of leukemia cells (Deininger et al. Blood, November 2000; 96(10): 3343-3356). This strategy can be used to restrict the replication of a given virus to a particular cell population or it can be used to restrict the function of a viral transcriptional regulator to restrict expression of a transgene in a particular cell population

### Example 3

Kinase-Dependent Inhibitory Domains/Subunits

A viral protein can have a negative regulatory domain fused to it that prevents the function of said protein unless the negative regulatory domain is phosphorylated. By designing the phosphorylation site on the negative regulatory domain to be a substrate for a specific cellular kinase one can restrict the function of this viral protein to those cells that express the particular kinase.

The inhibitory domain described above could be expressed from a separate gene in the recombinant viral genome and the resulting protein would bind to the target viral protein (and inhibit its function) unless phosphorylated.

In all cases above the phosphorylation event can be inhibitory or activating and regulate the replication of a given virus or regulate expression of a viral-encoded gene appropriately, depending on the presence or absence of a particular cellular kinase.

Specific Protease Restricted Viruses

### Example 4

Proteases from Chronically Virus Infected Cells

As an example, a virus such as vesicular stomatitis virus (VSV) that does not normally depend upon the activity of a virally coded protease can be engineered to become dependent upon specific proteolysis. As shown in FIG. 4A, VSV contains five genes that are all encoded by distinct mRNAs. As a result five separate proteins are independently synthesized. In FIG. 4A, the last two genes of VSV (G and L) are fused in such a way that only four viral mRNAs can be made from this genome. The fourth gene in this recombinant genome encodes a large polyprotein or “proprotein” containing both the G and L protein sequences. This fused protein contains at the G:L border, a protease recognition sequence which when cleaved liberates G and L as two functional proteins. The engineered protease site can only be recognized by a protease encoded by the HIV virus. As a result, productive infections of VSV can only occur in cells expressing the HIV protease or in other words cells chronically infected with HIV. Since VSV replication ultimately leads to the death of an infected cell, this virus will be able to specifically replicate in and kill HIV infected cells. This strategy could be extended to any cell that is chronically infected with a virus, an intracellular bacterium such as salmonella or shigella, a mycoplasm, or other intracellular parasite that encodes a specific protease (e.g. Hepatitis C-, Bovine Viral Diarrheal Virus, and Herpes-infected cells). Other examples are human T cell leukemia virus (HTLV), adenovirae, picornaviridae, calciviridae including Hepatitis E virus, and flaviviridae including dengue virus. Examples of intracellular parasites are: trypanosoma cruzi, mycobacterium tuberculosis and Plasmodium falciparum.

In Example 4A (FIG. 4D) the Hep C NS3/4A Protease Works in trans. 293T cells were transiently transfected with a test substrate (consisting of an inframe fusion between NS5A/5B/EGFP such that the natural Hap C cleavage site is present at the 5A/5B junction) and the NS3/4A protease. The western blot shown in FIG. 4D was probed with an anti-GFP antibody. The test substrate is cleaved (as indicated by the presence of the lower band) only in the presence of the co-transfected Hep C NS3/4A protease indicating that this protease can cleave this target sequence in Example 4B relates to the design of Hep C NS 3/4A Protease Dependent Recombinant VSV Genomes. FIG. 4E is a schematic of wildtype VSV genome with 5 individual genes coding for 5 separate viral proteins. FIG. 4F is a schematic of recombinant VSV genome where P and M open reading frames (ORFs) have been fused to code for a polyprotein containing the cleavage site for the Hep C NS3/4 protease (black box). FIG. 4G is a schematic of recombinant VSV genome where G and L ORFs have been fused to code for a polyprotein containing the cleavage for the Hep C NS3/4A protease (black box). FIG. 4H illustrates sequences for fusion junctions with the middle sequence in each case representing the mutant genome where the underlined sequence is from VSV and the intervening sequence codes for the cleavage site of the Hep C NS3/4A protease. The top and bottom sequences in each case are the oligos used to engineer these in-frame fusions.

Example 4C relates to a design of recombinant coxsackie B genomes engineered to have HepC NS3/4A cleavage sites. Such recombinant viruses would be dependent on the HepC protease function suplied in trans within a coinfected cell. FIG. 4I is a schematic of coxsackie virus polyprotein. Arrows indicate cleavage sites for coxsackie 3C protease (open) and coxsackie 2A protease (checkered). Individual coxsackie mutants were made with indicated coxsackie cleavage sites changed to Hep C NS3/4A cleavage site. FIG. 4J shows sequences for engineered cleavage sites. Middle sequence represent the engineered sequence with the flanking coxsackie sequence underlined and the introduced Hep C cleavage site intervening. Top and bottom sequences are for primers used to generate mutations.

### Example 5

Specific Protease Cleavage of Ubiquitin Fusion Proteins

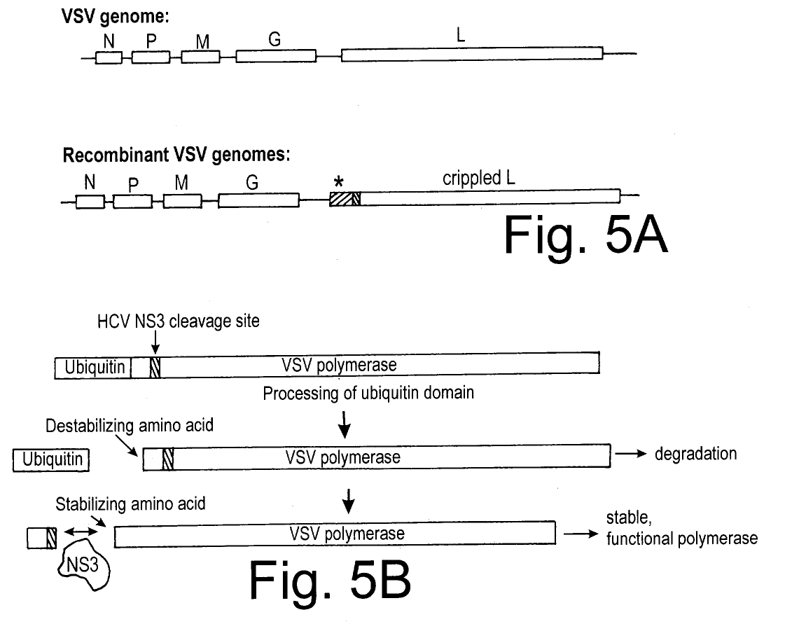

An alternative strategy is to alter an essential viral gene such that it is expressed as a fusion protein where an inhibitory domain is appended to the viral protein and prevents the function of said protein. A specific protease recognition sequence/cleavage site is placed within the fusion protein between the inhibitory domain and the viral protein sequence such that cleavage of the fusion protein by this specific protease yields a functional viral protein free of the inhibitory domain. Possible inhibitory domains include, but are not restricted to, domains which interact with the viral protein in an inhibitory fashion, domains which direct the fusion protein to be degraded in while or in part and domains which direct the fusion protein to a subcellular location where it is unable to perform its proper function. FIG. 5 describes a strategy to restrict the function of the VSV polymerase L to cells expressing a specific viral protease, NS3 of the hepatitis C virus (HCV). This “crippled L” is expressed as a fusion protein with ubiquitin (FIG. 5). When this fusion is expressed in the host cell the ubiquitin monomer would be cleaved from the fusion protein by the action of ubiquitin processing proteases (UBPs, Proc. Natl. Acad. Sci. U.S.A. Apr. 28, 1998; 95(9):5187-92), exposing a destabilizing amino-terminal amino acid. This directs the viral protein to be rapidly degraded by the host cell. The identity of this amino terminal amino acid determines the half-life of the modified viral protein and can be in the order of seconds to several minutes (reviewed in Varshavsky PNAS October 1996 93; 12142-49). In the presence of the HCV NS3 protease this recombinant viral proteins is rescued from rapid degradation as the viral protease cleaves the destabilized L protein yielding a different amino terminal amino acid that stabilizes the resulting, functional polymerase (FIG. 5). Through strategies such as this any viral protein can be made selectively unstable in cells that lack a particular protease required to remove a destabilizing amino terminus.

### Example 6

Stabilization of a Toxin

Alternatively, a toxin can be delivered by a virus or other means (liposome, HIV tat fusion protein, (Science. Sep. 3, 1999; 285(5433):1569-72)) that is conditionally stabilized by the action the HCV protease. Similarly to the model described above, a ubiquitin monomer can be fused to the amino terminus of a toxic protein followed by a HCV protease cleavage site. In HCV infected cells, the viral protease will selectively cleave the destabilizing residues from the toxin, resulting in the killing of the cell. In healthy cells, the toxin would be rapidly degraded by an N-end rule mechanism, following the removal of the ubquitin monomer.

### Example 7

Engineered Viruses with Novel Protease Sites

This strategy can also be used to make a virus that is normally dependent on its own protease to become dependent on a specific, heterologous protease expressed exclusively or predominantly in a particular cell population. An example would be the use of an adenoviral vector that is dependent upon the expression of the hepatitis C virus (HCV) protease NS3. Adenovirus is a particularly useful vector for targeting of liver cells and its replication is strictly dependent upon the expression of an adenovirus encoded protease. Indeed mutants of adenovirus that lack the protease are unable to grow except in cells, which constitutively express the viral protease (Hum. Gene Ther. Jun. 10, 2000; 11(9): 1341-53). For example, a mutant adenovirus that lacks the adenoviral protease but has been engineered to be susceptible to the HCV protease will replicate conditionally in cells expressing the HCV protease. This virus can be engineered to deliver a toxic or therapeutic protein only to cells expressing the HCV protease.

### Example 8

Prostate Specific Antigen Activation of Paramyxovirus Fusion Protein

Certain viruses including but not restricted to paramyxoviruses express viral envelope or surface proteins, which must be proteolytically cleaved before becoming activated. As an example the measles virus F protein must be cleaved by the ubiquitously expressed cellular protease furin in order to facilitate measles infection of cells. It is possible to alter the protease cleavage site on the F protein from a furin recognition site to a trypsin sensitive site and in so doing altering the cellular tropism of the recombinant virus (J. Gen. Virol. February 2000; 81 Pt2:441-9.)

Human prostate cancer cells are well known to secrete the protease PSA or prostate specific antigen. A pro-drug containing doxorubicin coupled to a peptide substrate of PSA has been developed. This pro-drug can be efficiently and specifically activated by the extra cellular enzyme PSA (Prostate. Sep. 15, 2000; 45(1):80-3.). An engineered paramyxovirus containing an F protein that can be activated by PSA mediated cleavage would be preferentially targeted to infect tumour cells.

Certain types of human diseases are characterized by robust expression of extra cellular proteases. For example, certain cancers express or locally activate matrix metalloproteinases (Cancer Lett. Mar. 13, 2000; 150(1):15-21.) An engineered paramyxovirus containing an F protein that can be activated by these metalloproteinases would be preferentially targeted to infect tumour cells.

### Example 9

Gene Therapy Vectors Dependent on Specific Proteases

Once again this strategy can be used not only to alter the tropism of a given recombinant virus but also to control the expression of a transgene encoded by a recombinant virus where the appropriate transactivating viral protein is modified to be a substrate of a specific protease and to be activated in the presence of said protease. In this case the replication of the recombinant virus may not be dependent on a specific host-expressed protease but the expression of viral transgenes would be. This strategy can use replication incompetent as well as competent viral vectors.

As well the transgene itself can be designed to be a substrate for a specific protease. In this case the transgene may be a “protoxin” which is itself a substrate for the specific protease and is thereby only activated in cells expressing the specific protease.

Selective Therapeutics Regulated by the Ubiquitin Degradative Pathway

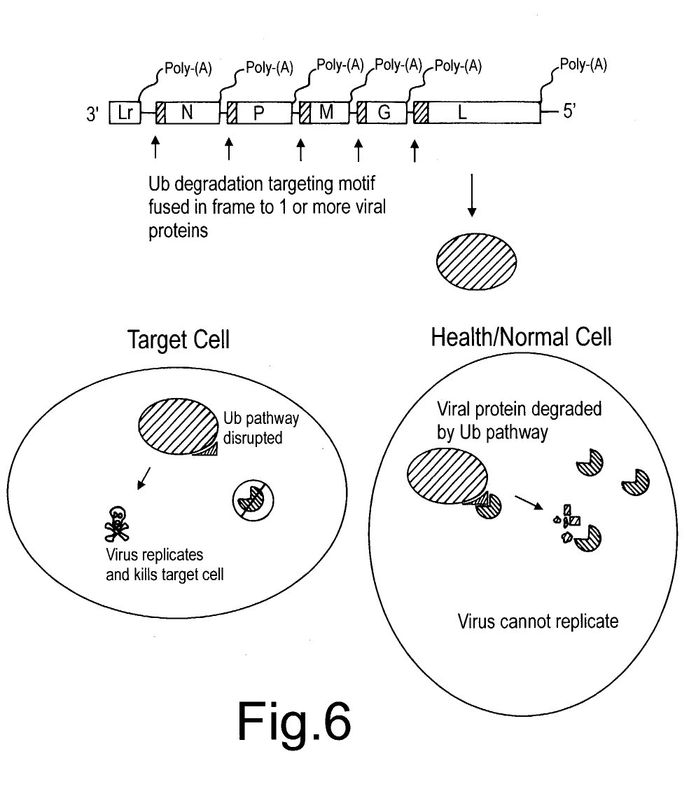

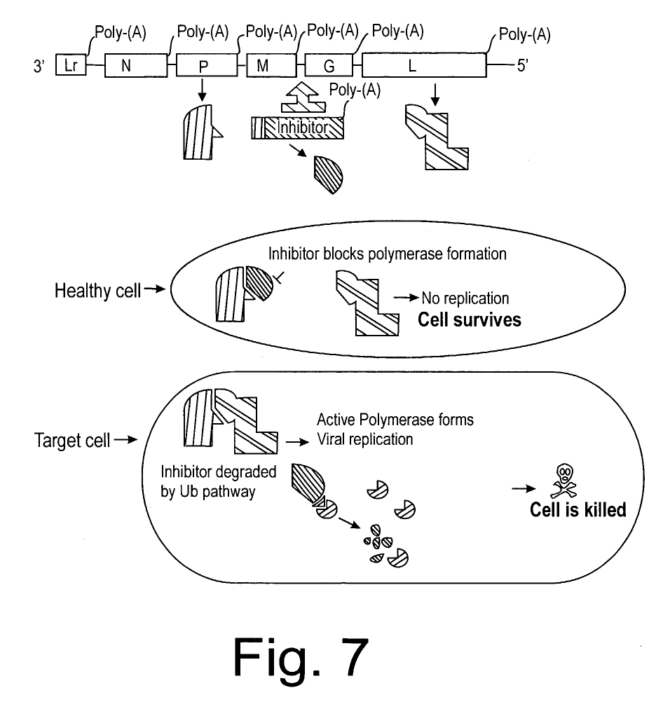

A virus (DNA or RNA) is engineered or identified from natural isolates, to be conditionally replicative; or conditionally deliver a prodrug; or deliver a prodrug which can be conditionally activated or inactivated under conditions regulated by components of the ubiquitin pathway. For example, one may engineer a virus such that a viral protein critical to replication, is selectively targeted for degradation via the ubiquitin pathway in a tissue/disease/stress specific manner (FIG. 6). This specific manner would be dependent on the tissue/disease/stress specific nature of one or more components of the ubiquitin pathway. Alternatively, a cytolytic virus could be constructed encoding an inhibitor of its own viral replication. This inhibitor could be designed to be targeted for Ub-mediated degradation in a tissue/disease/stress specific manner (FIG. 7). The inhibitor could be, but is not limited to, a dominant negative inhibitory of a viral polypeptide, a host antiviral defense proteins (PML, PKR, or other IFN inducible proteins) or a protease capable of degrading one or more viral proteins. The specific Ub-mediated degradation of this inhibitor in target cells would allow the cytolytic virus to replicate and kill the target cell. Non-target cells would not degrade the inhibitor and would therefore be protected from the virus.

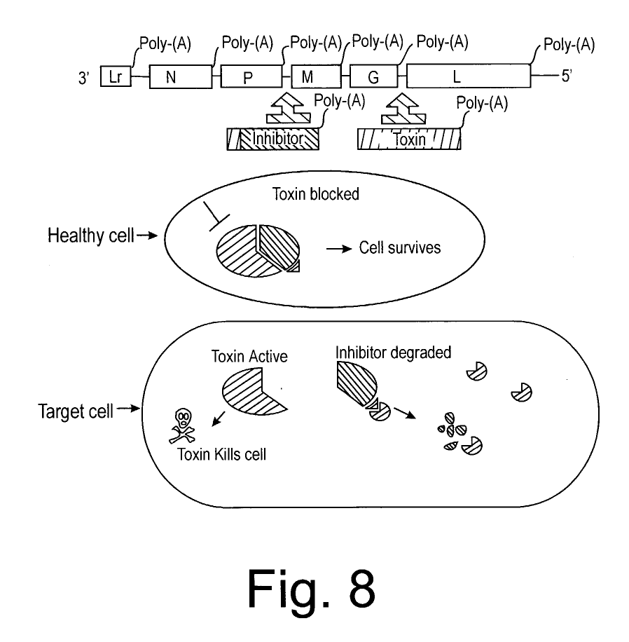

In another strategy, the virus could deliver a “pro-toxin” (FIG. 8). This pro-toxin could consist of two subunits, an active toxic subunit and a subunit which inhibits the toxin. The inhibitor subunit would be engineered to be selectively degraded by the Ub pathway in a target cell specific manner. Degradation of the toxin inhibitor would allow the toxin to kill the target cell. Healthy cells would not degrade the inhibitor and therefore would be protected from the toxin. Re-targeting of proteins for Ub mediated degradation has been described recently (Mol. Cell. September 2000; 6(3):751-6). In this case, the normally stable retinoblastoma protein was targeted for degradation by the re-engineering of an F-box protein, to selectively bind the Rb protein, facilitating its association with an E3 ligase complex and subsequent degradation.

### Example 10

Treatment of Virally Infected Cells Expressing a Ubiquitin Pathway Modifier

Human papilloma virus (HPV) expresses a protein (E6) which usurps the host E3 Ub ligase, E6 associated protein (E6-AP), to target the host tumour suppressor protein p53 (Proc. Natl. Acad. Sci. U.S.A. Jan. 30, 2001; 98(3):1218-1223). Cells infected with HPV will express the E6 protein. A cytolytic virus could be engineered to express an “inhibitor” of its own replication (see below). This inhibitor could be engineered with the amino acid motif, or optimized versions thereof, recognized by the E6/E6-AP complex (Proc. Natl. Acad. Sci. U.S.A. Jan. 30, 2001; 98(3):1218-1223) and thereby be selectively targeted for degradation by the Ub pathway in cells infected with HPV. The destruction of this inhibitor would allow replication and subsequent destruction of the HPV infected cell by the engineered virus.

Herpes Simplex Virus (HSV)ICP0 is required for HSV reactivation from quiescence in neurons (reviewed in Bioessays, August 2000; 22(8):761-70). HSV infected cells express ICP0. ICP0 has a ring finger domain and is required for the proteasome-dependent degradation of the ND10 protein Sp100 and other target proteins (Oncogene. Jan. 28, 1999; 18(4):935-41). HSV infected cells therefore could be selectively targeted for destruction by a cytolytic virus engineered to be conditionally replicative in cells expressing ICP0. For example, a “therapeutic virus” could be constructed encoding an inhibitor of its own replication. This inhibitor could be engineered to be selectively targeted for Ub mediated degradation by the direct or indirect actions of HSV ICP0 (FIG. 8). Upon infection of HSV infected cells with the “therapeutic virus”, the inhibitor would be degraded allowing replication of the cytolytic “therapeutic virus”, killing the HSV infected host cell. In uninfected (“HSV-free”) cells, where no ICP0 is expressed, the inhibitor would be stable and would block the replication of the “therapeutic virus”. This would protect uninfected “HSV-free” cells from destruction by the “therapeutic virus”.

Other examples are: BICPO in bovine herpes virus 1 (BHV-1); Eg63 in equine herpes virus 1 (EHV-1), Vg61 in varicella-zoster virus (VZV) and EPO in pseudorapies virus (PRV).

### Example 11

Viruses Which Grow in Cells With Defects in the Ubiquitin Pathway

In another embodiment, cells which have lost components of the Ub pathway, could be targeted for destruction. Several tumour suppressor genes have been characterized as components of the Ub pathway (Proc. Natl. Acad. Sci. U.S.A., Oct. 26, 1999; 96(22):12436-41, Nat. Genet. June 2000; 25(2):160-5). By engineering a cytopathic virus to be conditionally replicative in cells deficient in these components of the Ub pathway, one could selectively target these malignancies.

Von Hippel-Lindau disease is a dominant inherited syndrome characterized by the predisposition to develop various kinds of benign and malignant tumors, including clear cell renal carcinomas, pheochromocytomas, and hemangioblastomas of the central nervous system and retina (Medicine (Baltimore) November 1997; 76(6):381-91).

Inactivation or deletion of both alleles of the vhl gene was found in over 80% of sporadic clear cell renal carcinomas and cerebellar hemangioblastomas (Nat. Genet. May 1994; 7(1):85-90). The VHL gene product (pVHL) is a tumour suppressor protein which is known to be a component of the E3 Ub ligase complex containing elongin B, elongin C and cullin (CUL)-2 (Proc. Natl. Acad. Sci. U.S.A. Oct. 26, 1999; 96(22):12436-41). Naturally occurring pVHL mutations disrupt this E3 ligase complex. Furthermore, pVHL has been shown to, directly or indirectly, target hypoxia-inducible transcription factors, including hypoxia-inducible factor (HIF)-1 and HIF-2; for degradation by the 26S proteasome (Nature. May 20, 1999; 399(6733):271-5, J. Biol. Chem. Aug. 18, 2000; 275(33):25733-41). Presumably, tumour cells deleted in pVHL would therefore show a growth advantage under the hypoxic conditions normally associated with the tumour microenvironment.

A cytolytic virus could be engineered to be conditionally replicative in tumour cells deficient in pVHL activity. One or more of the virally encoded proteins required for replication could be engineered to include an amino acid sequence motif responsible for targeting proteins to an E3 ligase complex, via pVHL. In normal healthy cells, pVHL would target these proteins for Ub mediated degradation, and the virus would be unable to replicate and/or cause cytopathology. In tumour cells devoid of pVHL activity, the viral proteins would be stable, and the virus would replicate, leading to the destruction of the tumour cell (FIG. 6). Alternatively, or in combination with the above, a virus could be engineered to selectively deliver a toxin to tumour cells devoid of pVHL activity. To do so, this toxin could be engineered to include an amino acid sequence motif responsible for pVHL-mediated targeting to an E3 ligase complex. In healthy cells, the toxin would be degraded by the Ub pathway through the action of pVHL, while in pVHL deficient tumour cells, the toxin would remain stable and kill the cell.

Alternatively, regulators of tumour suppressors can also be components of the Ub pathway. The p53 tumour suppressor protein is targeted for degradation by the MDM2 E3 ligase (Oncogene. Mar. 9, 2000; 19(11):1473-6). The MDM2 oncogene is amplified or over expressed nimany human cancers. It also has been suggested that MDM2 levels are associated with poor prognosis of several human cancers (Curr Pharm. Des. March 2000; 6(4):393-416). This could result from any number of mechanisms including, but not restricted to, MDM2 gene amplification, or deletion or mutation of the p14 ARF MDM2 regulator protein. One study of primary human astrocytic gliomas reported 48% of glioblastomas, 13% of anaplastic astrocytomas, and 38% of astrocytomas had a deregulated p53 pathway either by amplification of MDM2, or homozygous deletion/mutation of p14ARF (Cancer Res. Jan. 15, 2000; 60(2):417-24).

A cytolytic virus could be engineered to express an “inhibitor” of its own replication (see below). This inhibitor could be engineered with the amino acid motif, or optimized versions thereof, recognized by the MDM2 E3 ligase (Proc. Natl. Acad. Sci. U.S.A. Jan. 30, 2001; 98(3):1218-1223) and thereby be selectively targeted for degradation by the Ub pathway in tumour cells with increased MDM2 activity. The destruction of this inhibitor would allow replication and subsequent destruction of the tumour cell by the engineered cytolytic virus.

### Example 12

Exploiting Stress Activated Ubiquitin Pathways

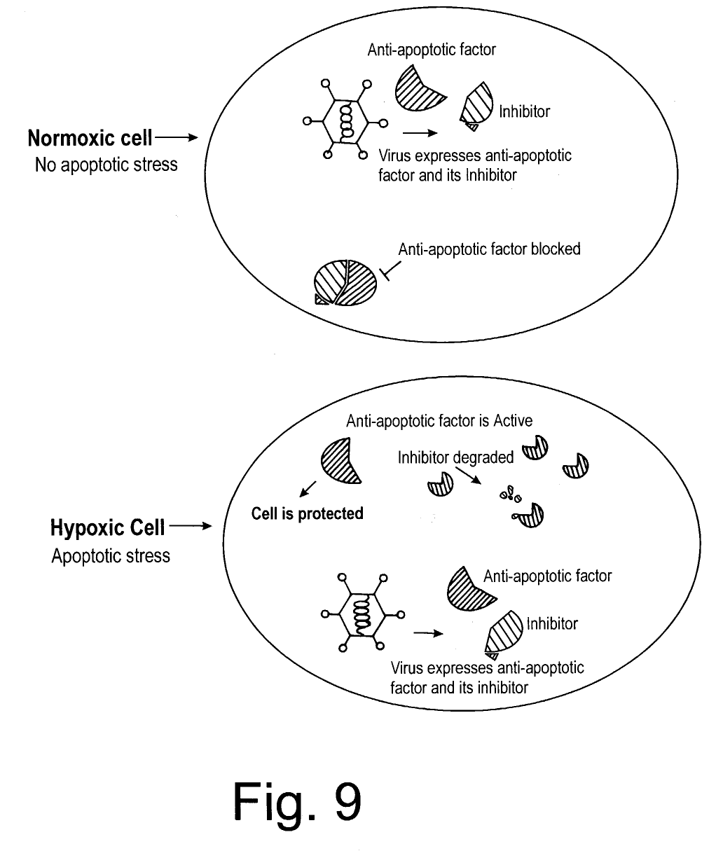

Ischemia is a condition where arteries become occluded or damaged, leading to inadequate oxygen to tissues due to a reduced or completely blocked blood supply. The two most common types of ischemia are cardiac and cerebral. In response to these hypoxic or anoxic conditions, a many gene products are up or down regulated by a variety of regulatory pathways, to protect the cell from damage (Pharm. Res. October 1999; 16(10):1498-505). At least one study demonstrates that hypoxia-elicited targeting of transcription factors for Ub-mediated proteasome-degradation represents one such adaptive response mechanism (Proc. Natl. Acad. Sci. U.S.A. Oct. 24, 2000; 97(22):12091-6). Ischemia, induced hypoxia, therefore, may be selectively treated by a virally delivered, or other, anti-apoptotic compound (Bcl-2, Exp. Cell. Res. Apr. 10, 2000; 256(1):50-7; Xiap. Genes Dev. Feb. 1, 1999; 13(3):239-52), conditionally responsive to alterations in the Ub degradative pathway of hypoxic cells. For example, a recombinant virus could be designed to persistently infect cells of the brain in a non-deleterious fashion. This virus would encode an anti-apoptotic protein as well as an inhibitor to this anti-apoptotic factor (FIG. 9). Hypoxia activates the proteolytic degradation of CREB via a 6 amino acid targeting motif (DSVTDS)(Proc. Natl. Acad. Sci. U.S.A. Oct. 24, 2000; 97(22):12091-6). This motif could be engineered into the coding sequence of the inhibitor protein, resulting in fusion protein which would be selectively degraded in hypoxic cells. Following degradation of the inhibitor, the anti-apoptotic protein would free to protect hypoxic cells from apoptosis mediated death. Healthy cells would not degrade the inhibitor, and would therefore not be subject to the effects of the anti-apoptotic protein.

The anti-apoptotic may be selected from members of the Bcl-2 family, including Bcl-2 and Bcl-xL; or members of the inhibitors of apoptosis family including xiap, cIAP1, cIAP2, and survivin.

Incorporation of Cellular Translational Regulatory Sequences into Viral Genomes

### Example 13

Linking of Cell Cycle Specific Translation to Viral Replication

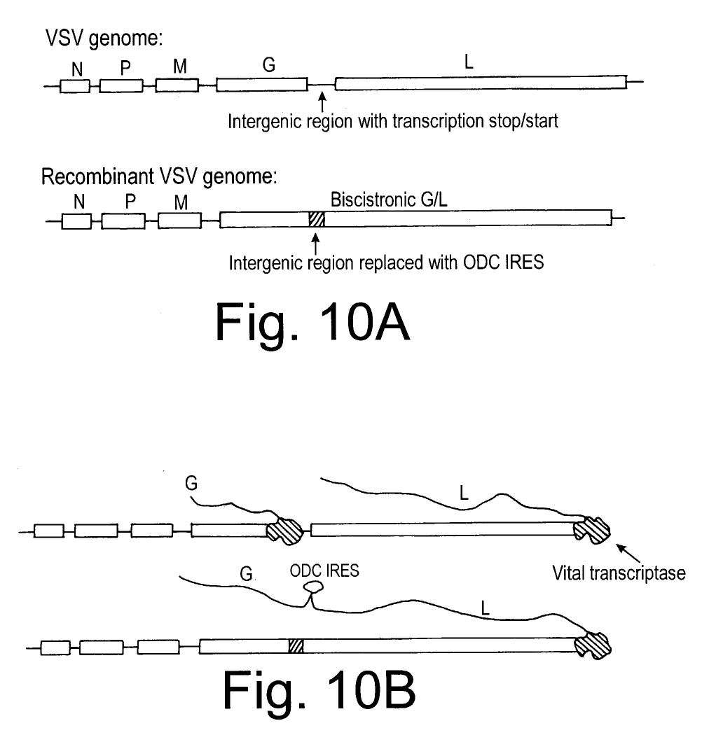

The ornithine decarboxylase (ODC) mRNA contains an internal regulatory element which facilitates the translation of this mRNA only during the mitotic cycle of cells (referred to as the ODC IRES see reference). This element has been shown to confer cell cycle specific translation to a reporter gene which is not normally regulated in this fashion. This element further has the ability to initiate cap independent translation. Vesicular stomatitis virus contains five genes which must be transcribed and translated in a defined fashion for efficient viral replication (see FIG. 10A). Normally these gene products can be translated in any cell type independent of the phase of the cell cycle, stage of development and under a broad range of physiological states (i.e. hypoxia). The intergenic region between the viral genes is required for initiation of transcripts with 5′ ends available for translation. As shown in FIG. 10A the intergenic region between the G gene and L gene is deleted and replaced by the ODC IRES. When this virus transcribes its genes, a novel transcript is produced which contains both the G and L coding regions linked by the ODC IRES. While the G cistron has a 5′ end that can be recognized by host cell translational machinery of most cell types, the L cistron lacks a free 5′ end and can only be translated during the G2/M transition. Thus in stationary non-dividing cells this virus will be unable to produce the L protein and thus its replication will be aborted (FIG. 10B). On the other hand, cells that are actively dividing will produce L protein and the replicative cycle program of the virus will proceed. Malignant cells are rapidly dividing cells and thus this virus will preferentially replicate in tumour cells but have impaired replication in normal non-dividing cells. Of particular interest, this virus would be unable to grow in non-dividing neuronal cells that are normally a target cell of VSV.

### Example 14

Use of CUG Initiator Codons

The FGF-2 mRNA contains multiple CUG initiator codons and a single AUG codon. In normal non-transformed cells, the CUGs are used inefficiently whereas in transformed tumour cells, the CUG initiator codons function efficiently even when transferred to a reporter mRNA (Cancer Res. Jan. 1, 1999; 59(1):165-71). In one series of recombinant viruses one or all of the AUG initiator codons for each viral gene will be replaced with portions of the FGF-2 leader sequences that confer CUG dependent initiation of translation. In another series of virus constructs each AUG initiator codon in one or more viral genes is replaced by a CUG codon.

### Example 15

5′ Terminal Oligopyrimidine Tracts

Stimulation of normal quiescent cells into mitosis induces recruitment of mRNAs containing at their 5′ termini short oligopyrimidine tracts known as TOPs. The ribosomal S6 kinase is responsible for the increased translation of TOPs containing mRNAs and is frequently found activated in malignant cells. In a series of recombinant virus constructs, a 5′-terminal oligopyrimidine tract is inserted onto one or more viral genes. The virus will be replicated preferentially in cells that can efficiently translate 5′-TOP containing mRNAs.

### Example 16

Translational Repressor Elements

The p53 mRNA contains a 66 nucleotide U rich element in its 3′UTR which when bound by p53 mediates translational repression in normal cells. Since p53 mutations are frequently found in human malignancies insertion of the 66-nucleotide repressor element into a viral mRNA will lead to increased viral mRNA translation in tumour cells compared to normal cells. In this example, the VSVL protein mRNA would be a preferred target as this enzyme is key to replication of the virus.

### Example 17

Upstream Open Reading Frames—uORFs

An upstream open reading frame is found in the transcription factor C/EBP. It functions to alter the re-initiation of protein synthesis at downstream AUG codons. The activity of the uORF is enhanced in cells that have increased elF-4E activity or decreased PKR activity, a common feature of malignant cells. Addition of the C/EBP uORF or analogous structures to a viral mRNA will affect the downstream AUG usage in that mRNA. In one application of this technology, the C/EBP uORF is placed upstream of the correct viral protein AUG at a distance that favours re-initiation of protein synthesis especially when eIF-4E activity is augmented.

### Example 18

5′ Structured Untranslated Regions

There is strong evidence that the increases in eIF-4E activity in tumour cells leads to increased translation in a small subset of proto-oncogene or growth promoting factors. Splicing of the 5′UTRs from the c-Myc, cyclin Dl, ornithine decarboxylase, basic fibroblast growth factor (FGF-2) and/or vascular endothelial growth factor (VEGF) onto some or all viral genes would lead to preferential virus replication in cells, which over express eIF-4E activity.

### Example 19

In another embodiment, cells which have lost components of the Ub pathway are targeted for destruction. Several tumour suppressor genes have been characterized as components of the Ub pathway (Proc Natl Acad Sci U.S.A. Oct. 26, 1999; 96(22):12436-41, Nat Genet June 2000; 25(2):160-5). By engineering a cytopathic virus to be conditionally replicative in cells deficient in these components of the Ub pathway, one could selectively target these malignancies.

Von Hippel-Lindau disease is a dominant inherited syndrome characterized by the predisposition to develop various kinds of benign and malignant tumors, including clear cell renal carcinomas, pheochromocytomas, and hemangioblastomas of the central nervous system and retina (Medicine (Baltimore) November 1997; 76(6):381-91). Inactivation or deletion of both alleles of the vhl gene was found in over 80% of sporadic clear cell renal carcinomas and cerebellar hemangioblastomas (Nat Genet May 1994; 7(1):85-90). The VHL gene product (pVHL) is a tumour suppressor protein which is known to be a component of the E3 Ub ligase complex containing elongin B, elongin C and cullin (CUL)-2 (Proc Natl Acad Sci USA. Oct. 26, 1999; 96(22):12436-41). Naturally occurring pVHL mutations disrupt this E3 ligase complex. Furthermore, pVHL has been shown to, directly or indirectly, target hypoxia-inducible transcription factors, including hypoxia-inducible factor (HIF)-1, for degradation by the 26S proteasome (Nature May 20, 1999; 399(6733):271-5, J Biol Chem Aug. 18, 2000; 275(33):25733-41). Presumably, tumour cells deleted in pVHL would therefore show a growth advantage under the hypoxic conditions normally associated with the tumour microenvironment.

A cytolytic virus could be engineered to be conditionally replicative in tumour cells deficient in pVHL activity. One or more of the virally encoded proteins required for replication could be engineered to include an amino acid sequence motif responsible for targeting proteins to an E3 ligase complex, via pVHL. In normal healthy cells, pVHL would target these proteins for Ub mediated degradation, and the virus would be unable to replicate and/or cause cytopathology. In tumour cells devoid of pVHL activity, the viral proteins would be stable, and the virus would replicate, leading to the destruction of the tumour cell. Alternatively, or in combination with the above, a virus could be engineered to selectively deliver a toxin to tumour cells devoid of pVHL activity (FIG. 6). To do so, this toxin could be engineered to include an amino acid sequence motif responsible for pVHL-mediated targeting to an E3 ligase complex. In healthy cells, the toxin would be degraded by the Ub pathway through the action of pVHL, while in pVHL deficient tumour cells, the toxin would remain stable and kill the cell.

Alternatively, regulators of tumour suppressors can also be components of the Ub pathway. The p53 tumour suppressor protein is targeted for degradation by the MDM2 E3 ligase (Oncogene Mar. 9, 2000; 19(11):1473-6). The MDM2 oncogene is amplified or overexpressed in many human cancers. It also has been suggested that MDM2 levels are associated with poor prognosis of several human cancers (Curr Pharm Des March 2000; 6(4):393-416). This could result from any number of mechanisms including, but not restricted to, MDM2 gene amplification, or deletion or mutation of the p14 ARF MDM2 regulator protein. One study of primary human astrocytic gliomas reported 48% of glioblastomas, 13% of anaplastic astrocytomas, and 38% of astrocytomas had a deregulated p53 pathway either by amplification of MDM2, or homozygous deletion/mutation of p14ARF (Cancer Res Jan. 15, 2000; 60(2):417-24).

A cytolytic virus could be engineered to express an “inhibitor” of its own replication (see below). This inhibitor could be engineered with the amino acid motif, or optimized versions thereof, recognized by the MDM2 E3 ligase (Proc Natl Acad Sci USA Jan. 30, 2001; 98(3):1218-1223) and thereby be selectively targeted for degradation by the Ub pathway in tumour cells with increased MDM2 activity. The destruction of this inhibitor would allow replication and subsequent destruction of the tumour cell by the engineered cytolytic virus.

Construction of Hypoxia Regulated Viral Vectors

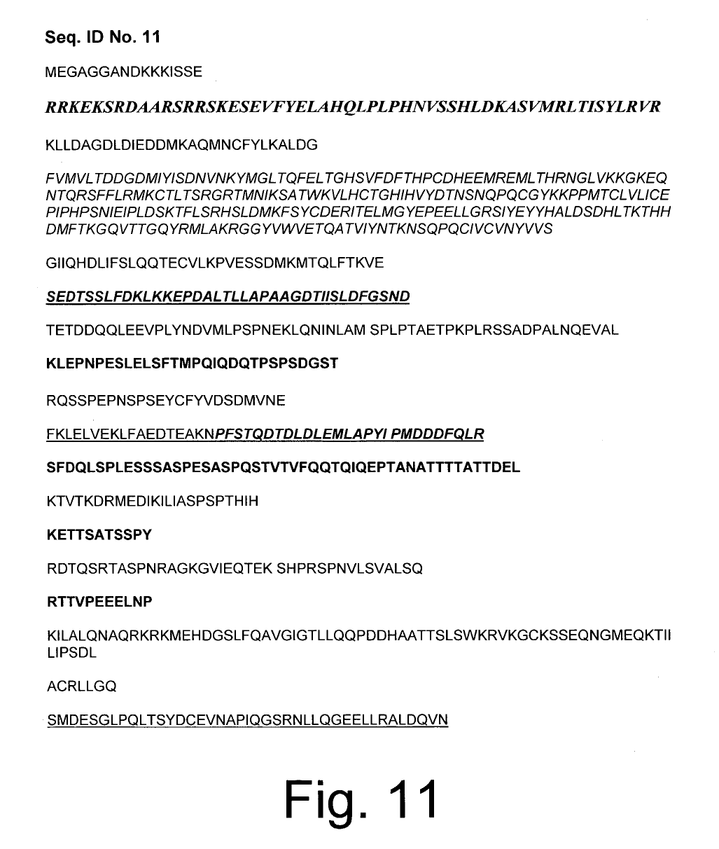

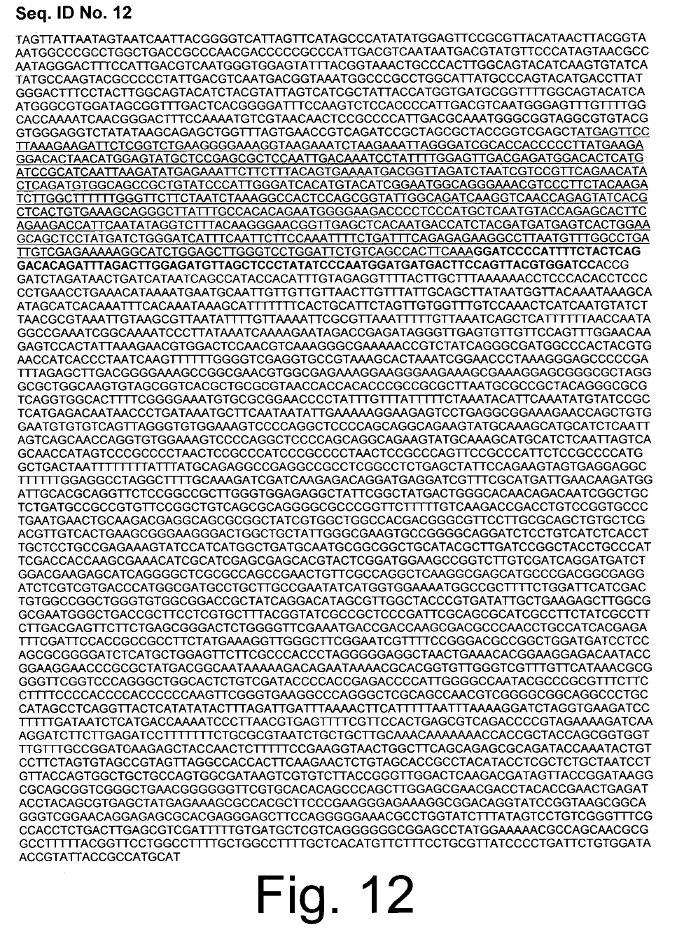

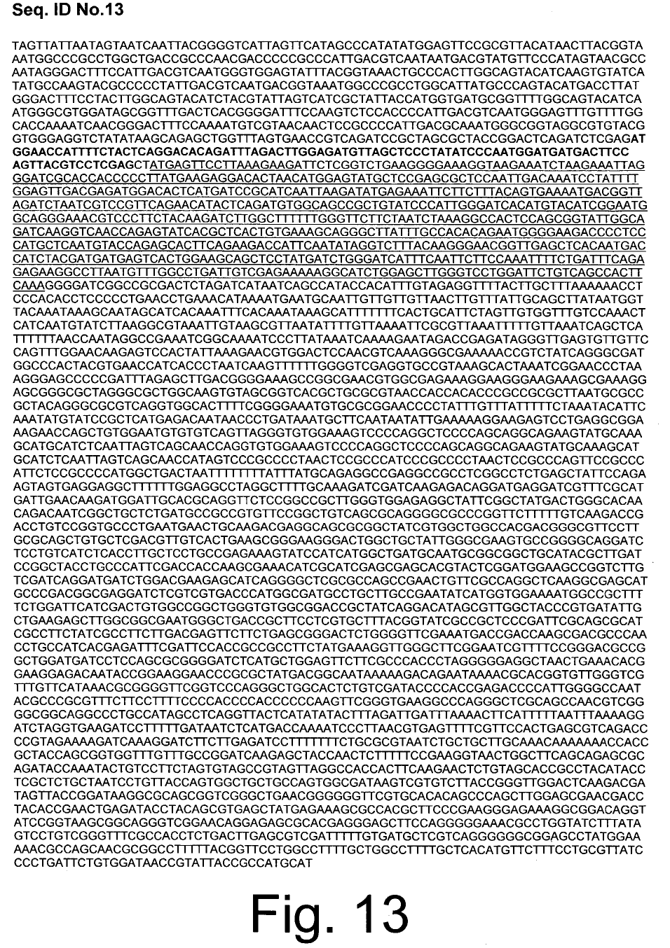

It has been well established that tumours are sites of hypoxia (Glatromanolaki and Harris 2001; Semenza 2002). Hif1α is a hypoxia induced protein whose protein stability is increased in hypoxic cells relative to cell under normoxic conditions. Under normoxic conditions, Hif1α is targeted for degradation via at least one of its two oxygen dependent degradation domains (ODDD)(Masson, William et al. 2001) (FIG. 11).

Structure Function of Hypoxia Inducible Factor 1 Alpha (HIF-1α) Protein.

In FIG. 11, the amino acid sequence of human Hif1-α has been annotated as per the various functional domains described in the literature. PAS domain (in italics only) implicated in protein:protein interactions (Semenza, Agani et al. 1997). Pest sequence in bold. Transactivation domains are underlined (Jiang, Zheng et al. 1997). Basic Heliz-loop-helix domain mediates DNA binding and protein dimerization (bold and italics)(Jiang, Rue et al. 1996). Oxygen dependent degradation domains 1 and 2 including the proline residue hydroxylase under normoxic conditions are indicated (bold italics and underlined) (Masson, William et al. 2001).

Hypoxic tumours therefore would contain cells which would have a diminished capacity to degrade Hif1α or perhaps proteins fused to Hif1α, or portions thereof.

As well, MDM2 also been reported to signal the degradation of HIF-1α in a p53 dependent manner (Ravi, Mookerjee et al. 2000). Since p53 mutations occur in 50% or more of all cancers (including over 50 tissue types) (Ravi, Mookerjee et al. 2000) it would follow that most cancer cells would be impaired in degrading Hif1α, or perhaps proteins fused to Hif1α, or portions thereof, containing the necessary regulatory sequences which render the protein stability of Hif1α dependent on p53 and/or MDM2.

Also, the protein phosphotase PTEN has been shown to negatively regulate the protein stability of Hif1α. Since deletion/mutation of the PTEN gene is very common in tumours (Cantley and Neel 1999) it would follow that a cancer cell would be impaired in its ability to degrade Hif1α or fusion proteins with all or portions of the Hif1α protein.

We propose the construction of viral vectors which encode gene products whose protein stability is regulated by the ubiquitin protein degradation pathway. Specifically, but not restricted to, the construction of replication competent viruses which code for proteins fused to the entire coding sequence, or fragments thereof, from the Hif1-α protein. For example, these fragments may include 1 or both of the oxygen dependent degadation domains (ODDD) which have been reported in the literature to bind VHL and target these proteins for degradation through the ubiquitin pathway (Masson, William et al 2001). The said fusion protein expressed in the virus may be any, of the endogenously coded viral proteins or maybe a heterologous protein. For example, if the virus was VSV, then each, or each in any combination, of the 5 endogenously coded proteins (N, P, M, G or L) could be fused to said ODDD containing fragment(s) to obtain a virus whose replication is dependent on the oxygen levels of the target cell.

FIG. 12 illustrates a minimal oxygen dependent degradation (ODDD) domain fused to the carboxy terminal coding sequence of VSV M protein. This vector was generated by cloning a full length VSV M PCR fragment into the BamHI and XhoI sites fo pEGFP-C1 followed by the insertion of a fragment of HIF1α representing the second ODDD fragment (produced from overlapping primer extension) into the XhoI site. Then the GFP coding sequence was removed from the vector by digesting with AgeI and XhoI sites, filling the religating. FIG. 5A shows the resulting sequence. HIF1αODDD fragment is designated in bold and the VSV M coding sequence is underlined.

FIG. 13 illustrates a minimal oxygen dependent degradation (ODDD) domain fused to the amino terminal coding sequence of VSV M protein. This vector was generated by cloning a full length VSV M PCR fragment into the BamHI and XhoI sites fo pEGFP-N1, followed by the insertion of a fragment of HIF1α representing the second ODDD fragment (produced from overlapping primer extension) into the BamHi site. Then the GFP coding sequence was removed from the vector by digesting with BamHI and NotI site, filling in the religating. HIF1αODDD fragment is designated in bold and the VSV M coding sequence is underlined.

The following are incorporated by reference into this disclosure:

Cantley, L. C. and B. G. Neel (1999). “New insights into tumor suppression: PTEN suppresses tumor formation by restraining the phosphoinositide 3-kinase/AKT pathway.” Proc Natl Acad Sci USA 96(8):4240-5.

The most recently discovered PTEN tumor suppressor gene has been found to be defective in a large number of human cancers. In addition, germ-line mutations in PTEN result in the dominantly inherited disease Cowden syndrome, which is characterized by multiple hamartomas and a high proclivity for developing cancer. A series of publications over the past year now suggest a mechanism by which PTEN loss of function results in tumors. PTEN appears to negatively control the phosphoinositide 3-kinase signaling pathway for regulation of cell growth and survival by dephosphorylating the 3 position of phosphoinositides.

Giatromanolaki, A. and A. L. Harris (2001). “Tumour hypoxia, hypoxia signaling pathways and hypoxia inducible factor expression in human cancer.” Anticancer Res 21(6B): 4317-24.

Hypoxia has been recognised as an important tumoral feature related to resistance to radiotherapy since 1933. Recent advances in biological research have revealed important aspects on the cellular response to hypoxic stimuli and on the role of hypoxia pathways in the metabolism, growth and progression of cancer. The hypoxia-inducible factors (HIF-1α and HIF-2α) have been identified as key proteins that directly respond to hypoxic stress. Following hypoxia, stabilisation and nuclear binding of HIFs triggers the expression of a variety of genes related to erythropoiesis, glycolysis and angiogenesis. This review reports on and discusses the biology of the hypoxia pathways, the studies performed on the expression of HIFs in human cancer and the implications of hypoxia pathways in cancer therapy.

Jiang, B. H., E. Rue, et al. (1996). “Dimerization, DNA binding, and transactivation properties of hypoxia-inducible factor 1,” J Biol Chem 271(30):17771-8.

Hypoxia-inducible factor 1 (HIF-1) is a heterodimeric basic helix-loop-helix transcription factor that regulates hypoxia-inducible genes including the human erythropoietin (EPO) gene. In this study, we report structural features of the HIF-1alpha and HIF-1beta (ARNT; aryl hydrocarbon receptor nuclear translocator) subunits were coimmunoprecipitated from nuclear extracts, indicating that these proteins heterodimerize in the absence of DNA. In vitro translated HIF-1alpha and HIF-1beta generated a HIF-1 complex with similar electrophoretic mobility and sequence specificity as HIF1 present in nuclear extracts from hypoxic cells. Compared to 826-amino acid, full-length HIF-1alpha, amino acids 1-166 mediated heterodimerization with HIF-1beta (ARNT), but amino acids 1-390 were required for optimal DNA binding. A deletion involving the basic domain of HIF-1alpha eliminated DNA binding without affecting heterodimerization. In cotransfection assays, forced expression of recombinant HIF-1alpha and HIF-1beta (ARNT) activated transcription of reporter genes containing EPO enhancer sequences with intact, but not mutant, HIF-1 binding sites. Deletion of the carboxy terminus of HIF-1alpha (amino acids 391-826) markedly decreased the ability of recombinant HIF-1 to activate transcription. Overexpression of a HIF-1alpha construct with deletions of the basic domain and carboxy terminus blocked reporter gene activation by endogenous HIF-1 in hypoxic cells.

Jiang, B. H., H. Z. Zheung, et al. (1997). “Transactivation and inhibitory domains of hypoxia-inducible factor 1alpha. Modulation of transcriptional activity by oxygen tension.” J Biol Chem 272(31):19253-60.

Hypoxia-inducible factor 1 (HIF-1) binds to cis-acting hypoxia response elements within the erythropoietin, vascular endothelial growth factor, and other genes to activate transcriptions in hypoxic cells. HIF-1 is a basic helix-loop-helix transcription factor composed of HIF-1alpha and HIF-1beta subunits. Here, we demonstrate that HIF-1alpha contains two transactivation domains located between amino acids 531 and 826. When expressed as GAL4 fusion proteins, the transcriptional activity of these domains increased in response to hypoxia. Fusion protein levels were unaffected by changes in cellular O2 tension. Two minimal transactivation domains were localized to amino acid residues 531-575 and 786-826. The transcriptional activation domains were separated by amino acid sequences that inhibited transactivation. Deletion analysis demonstrated that the gradual removal of inhibitory domain sequences (amino acids 576-785) was associated with progressively increased transcriptional activity of the fusion proteins, especially in cells cultured at 20% O2. Transcriptional activity of GAL4/HIF-1alpha fusion proteins was increased in cells exposed to 1% O2, cobalt chloride, or desferrioxamine, each of which also increased levels of endogenous HIF-1 alpha protein but did not affect fusion protein levels. These results indicate that increased transcriptional activity mediated by HIF-1 in hypoxic cells results from both increased HIF-1alpha protein levels and increased activity of HIF-1alpha transactivation domains.

Masson, N., C. William, et al. (2001). “Independent function of two destruction domains in hypoxia inducible factor-alpha chains activated by prolyl hydroxylation.” Embo J 20(18): 5197-206.

Oxygen-dependent proteolytic destruction of hypoxia-inducible factor-alpha (HIF-alpha) subunits plays a central role in regulating transcriptional responses to hypoxia. Recent studies have defined a key function for the von Hippel-Lindau tumour suppressor E3 ubiquitin ligase (VHLE3) in this process, and have defined an interaction with HIF-1 alpha that is regulated by prolyl hydroxylation. Here we show that two independent regions within the HIF-alpha oxygen-degradation domain (ODDD) are targeted for ubiquitylation by vHLE3 in a manner dependent upon prolyl hydroxylation. In a series of in vitro and in vivo assays, we demonstrate the independent and non-redundant operation of each site in regulation of the HIF system. Both sites contain a common core motif, but differ both in overall sequence and in the conditions under which they bind to the VHLE3 ligase complex. The definition of two independent destruction domains implicates a more complex system of pVHL-HIF-alpha interactions, but reinforces the role of prolyl hydroxylation as an oxygen-dependent destruction signal.

Ravi, R., B. Mookerjee, et al. (2000). “Regulation of tumor angiogenesis by p53-induced degradation of hypoxia-inducible factor 1alpha.” Genes Dev 14(1): 34-44.

The switch to an angiogenic phenotype is a fundamental determinant of neoplastic growth and tumor progression. We demonstrate that homozygous deletion of the p53 tumor suppressor gene via homologous recombination in a human cancer cell line promotes the neovascularization and growth of tumor xenografts in nude mice. We find that p53 promotes Mdm2-mediated ubiquitination and proteasonal degradation of the HIF-1alpha subunit of hypoxia-inducible factor 1 (HIF-1), a heterodimeric transcription factor that regulates cellular energy metabolism and angiogenesis in response to oxygen deprivation. Loss of p53 in tumor cells enhances HIF-1alpha levels and augments HIF-1-dependent transcriptional activation of the vascular endothelial growth factor (VEGF) gene in response to hypoxia. Forced expression of HIF-1alpha in p53-expressing tumor cells increases hypoxia-induced VEGF expression and augments neovascularization and growth of tumor xenografts. These results indicate that amplification of normal HIF-1-dependent responses to hypoxia via loss of p53 function contributes to the angiogenic switch during tumorigenesis.

Semenza, G. L. (2002). “Involvement of hypoxia-inducible factor 1 in human cancer.” Intern Med 41(2): 79-83.

Hypoxia-inducible factor 1 (HIF-1) mediates transcriptional responses to hypoxia. HIF-1 is composed of an O2- and growth factor-regulated HIF-1alpha subunit and a constitutively-expressed HIF-1beta subunit. Four lines of evidence indicate that HIF-1 contributes to tumor progression. First, HIF-1 controls the expression of gene products that stimulate angiogenesis, such as vascular endothelial growth factor, and promote metabolic adaptation to hypoxia, such as glucose transporters and glycolytic enzymes, thus providing a molecular basis for involvement of HIF-1 in tumor growth and angiogenesis. Second, in mouse xenograft models, tumor growth and angiogenesis are inhibited by loss of HIF-1 activity and stimulated by HIF-1alpha overexpression. Third, immunohistochemical analyses of human tumor biopsies indicate that HIF-1alpha is overexpressed in common cancers and that the level of expression is correlated with tumor grade, angiogenesis, and mortality. Fourth, in addition to intratumoral hypoxia, genetic alterations in tumor suppressor genes and oncogenes induce HIF-1 activity.

Semenza, G. L., F. Agani, et al. (1997). “Structural and functional analysis of hypoxia-inducible factor 1.” Kidney Int 51(2): 553-5.

Hypoxia-inducible factor 1 (HIF-1) is a basic helix-loop-helix protein that activates transcription of hypoxia-inducible genes, including those encoding: erythropoietin, vascular endothelial growth factor, heme oxygenase-1, inducible nitric oxide synthase, and the glycolytic enzymes aldolase A, enolase 1, lactate dehydrogenase A, phosphofruetokinase I, and phosphoglycerate kinase 1. Hypoxia response elements from these genes consist of a HIF-1 binding site (that contains the core sequence 5′-CGTG-3′) as well as additional DNA sequences that are required for function, which in some elements include a second HIF-1 binding site. HIF-1 is a heterodimer. The HIF-1 alpha subunit is unique to HIF-1, whereas HIF-1 beta (ARNT) can dimerize with other bHLH-PAS proteins. Structural analysis of HIF-1 alpha revealed that dimerization with HIF-1 beta (ARNT) requires the HLH and PAS domains, DNA binding is mediated by the basic domain, and that HIF-1 alpha contains a carboxyl terminal transactivation domain. Co-transfection of HIF-1 alpha and HIF-1 beta (ARNT) expression vectors and a reporter gene containing a wild-type hypoxia response element resulted in increased transcription in non-hypoxic cells and a superinduction of transcription in hypoxic cells, whereas HIF-1 expression vectors had no effect on the transcription of reporter genes containing a mutation in the HIF-1 binding site. HIF-1 alpha and HIF-1 beta (ARNT) protein levels were induced by hypoxia in all primary and transformed cell lines examined. In HeLa cells, the levels of HIF-1 alpha and HIF-1 beta protein and HIF-1 DNA-binding activity increased exponentially as cellular oxygen tension decreased, with maximum values at 0.5% oxygen and half-maximal values at 1.5 to 2% oxygen. HIF-1 alpha and HIF-1 beta (ARNT) mRNAs were detected in all human, mouse, and rat organs assayed and mRNA expression was modestly induced in rodents subjected to hypoxia. HIF-1 alpha protein levels were induced in vivo when animals were subjected to anemia or hypoxia. The HIF1A gene was mapped to human chromosome 14q21-q24 and mouse chromosome 12.

Combinatorial Application of Translational and Post-translational Regulatory Signals

This invention is not limited to the use of only one regulatory element in a particular virus construct. Combination of translational regulatory elements in a single viral construct may be preferred. For example, a CUG initiator codon may be placed in an IRES, which has been determined to work efficiently in cycling cells. The combination of these two elements will confer a further level of translational regulation upon a viral mRNA. It may be preferred to combine protease sensitivity, translational regulation or kinase dependency separately or together into a single viral construct to generate the optimum therapeutic.

Identification of Translational Regulatory Elements Useful in Design of Viral Vectors

While many elements are currently known it is likely that different types of elements exist that are more suitable for incorporation into therapeutic viruses. Identification of these elements can be accomplished by analyzing the polysome profiles in cells of different types. For instance comparing the content of mRNAs, which are enriched in polysomes of cells from different tissues, physiological states, developmental stages or disease states will identify mRNAs, which are differentially translated. As an example, polysomes isolated from quiescent and activated T cells will identify mRNAs that are preferentially translated in activated T cells. These mRNA molecules will contain unique regulatory elements that facilitate their translation. These elements may be in the 5′UTR, 3′UTR, in coding regions or function in concert. Removal of these elements from the cellular mRNA and inclusion into viral mRNA sequences will confer efficient replication of the virus in activated T cells. A virus with these properties could be used to treat autoimmune diseases.

The p53 gene product is a known regulator of translation and is frequently deleted in cancer cells. Using cells, which have inducible expression of p53, will allow identification of transcripts that are differentially translated between normal and malignant cells. Polysomes isolated from cells induced to express p53 (either mutant or wild type) would be compared.

Use of Recombinant Viruses to Isolate Inhibitors of Translation Regulatory Elements

A recombinant virus, which is dependent upon, incorporated cellular translation regulatory elements for its replication would be used to screen for inhibitors of said elements. For instance, a virus that relies upon the FGF-2 CUG initiator codon for its replication would be used to screen for inhibitors of the CUG dependent translation. As CUG codon usage is more efficient in tumour cells, this virus would be grown on tumor cells where it would selectively replicate. Adding an inhibitor of the translation regulatory would block virus replication. This assay could be automated to a 96 well format where susceptible cells are seeded into each well and then infected with recombinant virus in the presence of a panel of small molecule inhibitors. Where the virus grew unabated, the cells would be killed whereas in the presence of an effective inhibitor, the virus would not grow. The wells could be scored either for viable cells or the presence of viral particles or antigens.

Production of Conditionally Replicative Viruses

The production of recombinant viruses described in this application would be accomplished through the use of host cell lines which express the characteristic on which the replication of the virus depends. For example a virus which depends on the function of a particular kinase will be produced in a cell line engineered to over express this kinase. A virus which is dependent on a particular protease will be produced in a cell line engineered to express this particular protease. A virus with translational elements which are efficiently recognized in a particular cell type or in cells in a particular growth state etc. will be grown in cells where these elements will be efficiently utilized (i.e. a virus dependent on the ODC IRES will be grown in actively cycling cells).

1. A virus modified to contain in its genome, a viral gene comprising two separate open reading frames (ORFs), a first of said ORFs comprising a fusion with a sequence coding for a recognition sequence for a cellular kinase protein specific to a diseased cell, and a second of said ORFs comprising a fusion with a sequence coding for a protein which binds to said recognition sequence exclusively when said recognition sequence is phosphorylated, thereby reconstituting a viral protein, said reconstituted viral protein being essential to viral replication.

2. A modified virus as defined in claim 1, wherein said viral protein is essential to expression of virally encoded genes or transgenes.

3. A genetically modified virus comprising a gene sequence which codes for a mutated viral phosphoprotein having a phosphorylation site of a type which when non-mutated is phosphorylated by a cognate kinase, said phosphoprotein being capable of acting within a target cell such that said viral phosphoprotein is not phosphorylated by said cognate kinase, but rather is recognized solely by a kinase which is either restricted in its expression, or hyperactivated in a target cell, wherein phosphorylation of said viral phosphoprotein by said kinase is critical to viral replication thus restricting the replication of said virus to said target cell.

4. A virus as defined in claim 3, wherein said mutated viral phosphoprotein is a viral protein critical to expression of virally encoded genes or transgenes.

5. A virus as defined in claim 3, wherein said mutated viral phosphoprotein is the P protein of vesicular stomatitis virus (VSV).

6. A virus as defined in claim 3, wherein said phosphorylation site is recognized by a kinase which is overexpressed or hyperactivated in malignant cells.

7. A virus as defined in claim 6, wherein said kinase includes Akt/PKB, MAP kinase, BCR/ABL and TEL/ABL.

8. A genetically engineered virus containing a viral gene expressed as a fusion protein including: an inhibitory domain appended to a viral protein, said inhibitory domain for preventing the function of said fusion protein: a specific protease cleavage site between said inhibitory domain and said viral protein sequence for cleavage of said fusion protein by a specific protease contained within a target cell to yield a functional viral protein free of said inhibitory domain.

9. A virus as defined in claim 8, wherein said inhibitory domain is for destabilizing said viral protein sequence.

10. A virus as defined in claim 9, wherein said viral protein sequence includes a ubiquitin (Ub) monomer.

11. A virus as defined in claim 8, wherein said inhibitory domain is for directing said viral fusion protein to an inappropriate subcellular compartment.

12. A virus as defined in claim 8, wherein said inhibitory domain is fused to a viral protein and is for inhibiting said viral protein, said viral protein being critical to viral replication.

13. A virus as defined in claim 8, wherein said inhibitory domain is fused to a viral protein and is for inhibiting said viral protein, said viral protein being critical to the expression of a viral transgene.

14. A genetically modified virus containing a plurality of cistrons, at least two of said cistrons being linked by a nucleotide sequence acting as an internal ribosome entry site (IRES) element, said IRES element being exclusively or preferentially active in a target cell population in such a way that the second of said two linked cistrons is converted to a protein product only in said target cell.

15. A genetically modified virus as defined in claim 14, wherein said second of said two linked cistrons encodes a protein product critical to the replicative cycle of said virus.

16. A genetically modified virus as defined in claim 14, wherein the replication of said virus is toxic to said target cell.

17. A genetically modified virus as defined in claim 14, wherein said second of said two linked cistrons encodes a protein product, said protein product being toxic to said target cell.

18. A genetically modified virus as defined in claim 14, wherein said second of said two linked cistrons encodes a protein product, said protein product being therapeutic to said target cell.

19. A genetically modified virus as defined in claim 14, wherein said IRES element is exclusively or preferentially active in dividing cells.

20. A virus as defined in claim 14, wherein said IRES element is derived from the ornithine decarboxylase mRNA 5′ untranslated region.

21. A genetically modified virus as defined in claim 14, wherein said IRES element is active only in stressed cells, said stressed cells including hypoxic cells.

22. A genetically modified virus as defined in claim 14, wherein said IRES element is active only in said target cell, said target cell being an activated T cell.


SEQUENCE LISTING (14 sequences)

SEQ ID NO: 1 - protein, 265 aa, Rhabdoviridae vesiculoris (VSV Indiana strain)
  ref <301>: AUTHORS: Patnaik et al.
  ref <303>: JOURNAL: Journal of Virology
  ref <304>: VOLUME: 71
  ref <306>: PAGES: 8167 - 75
  ref <307>: DATE: 1997
MDNLTKVREYLKSYSRLDQAVGEIDEIEAQRAEKSNYELFQEDGVEEHTKPSYFQAADDSDTESEPEIEDNQGLYAQDPEAEQVEGFIQGPLDDYADEEVDVVFTSDWKPPELESDEHGKTLRLTSPEGLSGEQKSQWLSTIKAVVQSAKYWNLAECTFEASGEGVIMKERQITPDVYKVTPVMNTHPSQSEAVSDVWSLSKTSMTFQPKKASLQPLTISLDELFSSRGEFISVGGDGRMSHKEAILLGLRYKKLYNQARVKYSL

SEQ ID NO: 2 - protein, 265 aa, Rhabdoviridae vesiculoris (VSV Indiana strain)
MDNLTKVREYLKSYSRLDQAVGEIDEIEAQRAEKSNYELFQEDGVEEHTKPSYFRARDRSRTESEPEIEDNQGLYAQDPEAEQVEGFIQGPLDDYADEEVDVVFTSDWKPPELESDEHGKTLRLTSPEGLSGEQKSQWLSTIKAVVQSAKYWNLAECTFEASGEGVIMKERQITPDVYKVTPVMNTHPSQSEAVSDVWSLSKTSMTFQPKKASLQPLTISLDELFSSRGEFISVGGDGRMSHKEAILLGLRYKKLYNQARVKYSL

SEQ ID NO: 3 - protein, 265 aa, Rhabdoviridae vesiculoris (VSV Indiana strain)
  Mutagen 63: Xaa is any amino acid other than Asp or Glu
  Mutagen 67: Xaa is any amino acid other than Asp or Glu
MDNLTKVREYLKSYSRLDQAVGEIDEIEAQRAEKSNYELFQEDGVEEHTKPSYFRARDRSRTXSEPXIEDNQGLYAQDPEAEQVEGFIQGPLDDYADEEVDVVFTSDWKPPELESDEHGKTLRLTSPEGLSGEQKSQWLSTIKAVVQSAKYWNLAECTFEASGEGVIMKERQITPDVYKVTPVMNTHPSQSEAVSDVWSLSKTSMTFQPKKASLQPLTISLDELFSSRGEFISVGGDGRMSHKEAILLGLRYKKLYNQARVKYSL

SEQ ID NO: 4 - DNA, 82 bp, Flaviridae hepacivirus (hep C)
  Misc_feature 21-62: Mutant genome containing a cleavage site of the hep C NS3/4A protease
ggcgagagtcaaatattctgggggccgacacggaagatgtcgtgtgctgctcaatgtcttatatgagttccttaaagaagat

SEQ ID NO: 5 - DNA, 82 bp, Flaviridae hepacivirus (hep C)
  Misc_feature 21-62: Mutant genome containing a cleavage site of the hep C NS3/4A protease
agatgaaccgacttggaaagggggccgacacggaagatgtcgtgtgctgctcaatgtcttatatggaagtccacgattttga

SEQ ID NO: 6 - DNA, 82 bp, Flaviridae hepacivirus (hep C)
  Misc_feature 21-62: Mutant genome containing a cleavage site of the hep C NS3/4A protease
agcactactttaatgatgagggggccgacacggaagatgtcgtgtgctgctcaatgtcttatcacatcgagttcatcgaaag

SEQ ID NO: 7 - DNA, 82 bp, Flaviridae hepacivirus (hep C)
  Misc_feature 21-62: Mutant genome containing a cleavage site of the hep C NS3/4A protease
cgacccttgaggcgctattcggggccgacacggaagatgtcgtgtgctgctcaatgtcttatcccccagtgtacagagaaat

SEQ ID NO: 8 - DNA, 82 bp, Flaviridae hepacivirus (hep C)
  Misc_feature 21-62: Mutant genome containing a cleavage site of the hep C NS3/4A protease
gaatacccatggctgaacgcggggccgacacggaagatgtcgtgtgctgctcaatgtcttataacgggtggctaaagaaatt

SEQ ID NO: 9 - DNA, 82 bp, Flaviridae hepacivirus (hep C)
  Misc_feature 21-62: Mutant genome containing a cleavage site of the hep C NS3/4A protease
tggaagatgatgcaatggaaggggccgacacggaagatgtcgtgtgctgctcaatgtcttatgtgaaagattacgttgagca

SEQ ID NO: 10 - DNA, 82 bp, Flaviridae hepacivirus (hep C)
  Misc_feature 21-62: Mutant genome containing a cleavage site of the hep C NS3/4A protease
ttaagcaaacaaacttttatggggccgacacggaagatgtcgtgtgctgctcaatgtcttatgtgaaagattacgttgagca

SEQ ID NO: 11 - DNA, 82 bp, Flaviridae hepacivirus (hep C)
  Misc_feature 21-62: Mutant genome containing a cleavage site of the hep C NS3/4A protease
gcttgataaccacaggccccggggccgacacggaagatgtcgtgtgctgctcaatgtcttatcatcaatcaggggccgtgta

SEQ ID NO: 12 - DNA, 4727 bp, Homo sapiens; Rhabdoviridae vesiculoris (VSV Indiana strain)
  CDS (612)..(1297): r. Vesiculovirus M protein 1298-1403: hif1 Alpha ODDD
  CDS (1298)..(1403): hif1 Alpha ODDD
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtcgagctatgagttccttaaagaagattctcggtctgaaggggaaaggtaagaaatctaagaaattagggatcgcaccacccccttatgaagaggacactaacatggagtatgctccgagcgctccaattgacaaatcctattttggagttgacgagatggacactcatgatccgcatcaattaagatatgagaaattcttctttacagtgaaaatgacggttagatctaatcgtccgttcagaacatactcagatgtggcagccgctgtatcccattgggatcacatgtacatcggaatggcagggaaacgtcccttctacaagatcttggcttttttgggttcttctaatctaaaggccactccagcggtattggcagatcaaggtcaaccagagtatcacgctcactgtgaaagcagggcttatttgccacacagaatggggaagacccctcccatgctcaatgtaccagagcacttcagaagaccattcaatataggtctttacaagggaacggttgagctcacaatgaccatctacgatgatgagtcactggaagcagctcctatgatctgggatcatttcaattcttccaaattttctgatttcagagagaaggccttaatgtttggcctgattgtcgagaaaaaggcatctggagcttgggtcctggattctgtcagccacttcaaaggatccccattttctactcaggacacagatttagacttggagatgttagctccctatatcccaatggatgatgacttccagttacgtggatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 13 - DNA, 4740 bp, Homo sapiens; Rhabdoviridae vesiculoris (VSV Indiana strain)
  CDS (619)..(711): hif1 Alpha ODDD
  CDS (712)..(1401): 712-1401: r. Vesiculovirus M protein
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagatggaaccattttctactcaggacacagatttagacttggagatgttagctccctatatcccaatggatgatgacttccagttacgtcctcgagctatgagttccttaaagaagattctcggtctgaaggggaaaggtaagaaatctaagaaattagggatcgcaccacccccttatgaagaggacactaacatggagtatgctccgagcgctccaattgacaaatcctattttggagttgacgagatggacactcatgatccgcatcaattaagatatgagaaattcttctttacagtgaaaatgacggttagatctaatcgtccgttcagaacatactcagatgtggcagccgctgtatcccattgggatcacatgtacatcggaatggcagggaaacgtcccttctacaagatcttggcttttttgggttcttctaatctaaaggccactccagcggtattggcagatcaaggtcaaccagagtatcacgctcactgtgaaagcagggcttatttgccacacagaatggggaagacccctcccatgctcaatgtaccagagcacttcagaagaccattcaatataggtctttacaagggaacggttgagctcacaatgaccatctacgatgatgagtcactggaagcagctcctatgatctgggatcatttcaattcttccaaattttctgatttcagagagaaggccttaatgtttggcctgattgtcgagaaaaaggcatctggagcttgggtcctggattctgtcagccacttcaaaggggatcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 14 - protein, 826 aa, homo Sapiens
  ref <301>: AUTHORS: Wang et al.
  ref <303>: JOURNAL: Proceedings of the National Academy of Sciences
  ref <304>: VOLUME: 92
  ref <306>: PAGES: 5510-5514
  ref <307>: DATE: 1995
MEGAGGANDKKKISSERRKEKSRDAARSRRSKESEVFYELAHQLPLPHNVSSHLDKASVMRLTISYLRVRKLLDAGDLDIEDDMKAQMNCFYLKALDGFVMVLTDDGDMIYISDNVNKYMGLTQFELTGHSVFDFTHPCDHEEMREMLTHRNGLVKKGKEQNTQRSFFLRMKCTLTSRGRTMNIKSATWKVLHCTGHIHVYDTNSNQPQCGYKKPPMTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWVETQATVIYNTKNSQPQCIVCVNYVVSGIIQHDLIFSLQQTECVLKPVESSDMKMTQLFTKVESEDTSSLFDKLKKEPDALTLLAPAAGDTIISLDFGSNDTETDDQQLEEVPLYNDVMLPSPNEKLQNINLAMSPLPTAETPKPLRSSADPALNQEVALKLEPNPESLELSFTMPQIQDQTPSPSDGSTRQSSPEPNSPSEYCFYVDSDMVNEFKLELVEKLFAEDTEAKNPFSTQDTDLDLEMLAPYIPMDDDFQLRSFDQLSPLESSSASPESASPQSTVTVFQQTQIQEPTANATTTTATTDELKTVTKDRMEDIKILIASPSPTHIHKETTSATSSPYRDTQSRTASPNRAGKGVIEQTEKSHPRSPNVLSVALSQRTTVPEEELNPKILALQNAQRKRKMEHDGSLFQAVGIGTLLQQPDDHAATTSLSWKRVKGCKSSEQNGMEQKTIILIPSDLACRLLGQSMDESGLPQLTSYDCEVNAPIQGSRNLLQGEELLRALDQVN